(19) United States
(12) Patent Application Publication
(10) Pub. No.: US 2025/0188123 A1
(43) Pub. Date: Jun. 12, 2025

(54) PROTEASE INHIBITORS

(71) Applicants: Regents of the University of Minnesota, Minneapolis, MN (US); Universitat Regensburg, Regensburg (DE)

(72) Inventors: Michael A. Walters, Minneapolis, MN (US); Karen Ashe, Minneapolis, MN (US); Dezhi Liao, Minneapolis, MN (US); Gurpreet Singh, Minneapolis, MN (US); Merlin Bresinsky, Regensburg (DE); Alexander Hubmann, Regensburg (DE); Steffen Pockes, Regensburg (DE)

(21) Appl. No.: 18/840,842

(22) PCT Filed: Feb. 27, 2023
(86) PCT No.: PCT/US2023/013993

§ 371 Date: Aug. 22, 2024

Related U.S. Application Data
(60) Provisional application No. 63/313,862, filed on Feb. 25, 2022.

(51) Int. Cl. C07K 7/06; A61K 38/00; A61P 25/28; C07K 7/02

(52) U.S. Cl. CPC C07K 7/06; A61P 25/28; C07K 7/02; A61K 38/00

(57) ABSTRACT

The invention provides a compound of Formula (I): or a salt thereof, wherein R¹-R⁷, X, Y, and R^{a}-R^{z} have any of the values described in the specification, as well as compositions comprising a compound of Formula (I). The compounds are useful as caspase-2 inhibitors.

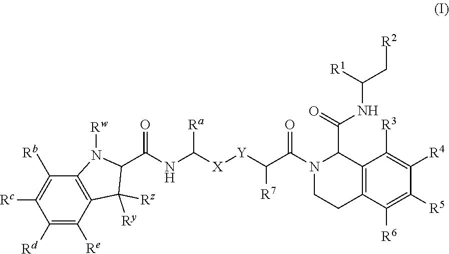

### CROSS-REFERENCE TO RELATED APPLICATION

This application claims priority to U.S. Provisional Application No. 63/313,862 that was filed on Feb. 25, 2022. The entire content of the application referenced above is hereby incorporated by reference herein.

### STATEMENT REGARDING FEDERALLY SPONSORED RESEARCH

This invention was made with government support under AG062199 awarded by National Institutes of Health. The government has certain rights in the invention.

### BACKGROUND

Currently there is a need for therapeutic agents that are Caspase-2 inhibitors. Such agents would be useful for treating neurodegenerative diseases, liver diseases, and cognitive dysfunction. There is also a need for molecular probes that bind to Caspase-2. These probes would be useful tools for investigating Caspase-2 biology and pharmacology.

### SUMMARY

A series of compounds that bind to caspase-2 have been identified. In one embodiment, the compounds are selective for caspase-2 over other biological targets (e.g., other caspases). In another embodiment, the compounds are reversible caspase-2 inhibitors. In another embodiment, the compounds possess a high level of brain penetration. In one embodiment, the compound includes a detectable group, so that the compound can be used as a detectable probe for studying Caspase-2 biology and pharmacology.

Accordingly, in one embodiment, the invention provides a compound of the invention, which is a compound of Formula (I):

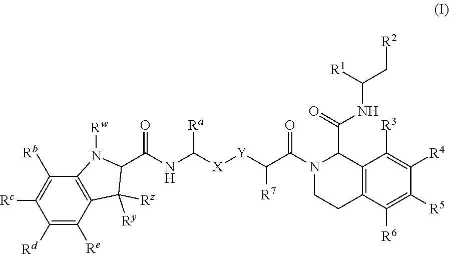

or a salt thereof, wherein:
R¹ is —B(OH)₂, cyano, (C₁-C₆)alkyl, (C₂-C₆)alkenyl, or (C₂-C₆)alkynyl, wherein each (C₁-C₆)alkyl, (C₂-C₆)alkenyl, and (C₂-C₆)alkynyl is optionally substituted with one or more groups independently selected from the group consisting of halo, cyano, oxo, NR^{r}R^{s}, and aryl, wherein each aryl is optionally substituted with one or more groups independently selected from the group consisting of halo, hydroxy, (C₁-C₆)alkyl, cyano, (C₁-C₆)alkoxy, (C₁-C₆)alkoxycarbonyl, carboxy, and (C₁-C₆)alkanoyloxy, and wherein one or more sp3 carbon atoms of each (C₁-C₆)alkyl, (C₂-C₆)alkenyl, and (C₂-C₆)alkynyl is optionally replaced with O, S, S(O), S(O)₂, or NH; or R¹ is —C(═O)R^{p};
R² is —C(═O)OR^{f} or —C(═O)NR^{g}R^{h};
R³ is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R⁴ is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R⁵ is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R⁶ is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R⁷ is H or (C₁-C₆)alkyl that is optionally substituted with (C₁-C₆)alkoxy or (aryl)(C₁-C₆)alkoxy;
R^{a} is H or (C₁-C₆)alkyl that is substituted with —C(═O)OR^{k} or —C(═O)NR^{m}Rⁿ;
R^{b} is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy R^{c} is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy
R^{d} is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R^{e} is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy
R^{f} is selected from the group consisting of H, (C₁-C₆)alkyl, (C₃-C₆)cycloalkyl, (C₃-C₆)cycloalkyl(C₁-C₆)alkyl, and aryl(C₁-C₆);
each R^{g} and R^{h} is independently selected from the group consisting of H, (C₁-C₆)alkyl, (C₃-C₆)cycloalkyl, (C₃-C₆)cycloalkyl(C₁-C₆)alkyl, and aryl(C₁-C₆); or R^{g} and R^{h} taken together with the nitrogen to which they are attached form an aziridino, azetidino, morpholino, piperazino, pyrrolidino or piperidino, which aziridino, azetidino, morpholino, piperazino, pyrrolidino or piperidino is optionally substituted with one or more groups independently selected from the group consisting (C₁-C₆)alkyl;
R^{k} is selected from the group consisting of H, (C₁-C₆)alkyl, (C₃-C₆)cycloalkyl, (C₃-C₆)cycloalkyl(C₁-C₆)alkyl, and aryl(C₁-C₆);
each R^{m} and Rⁿ is independently selected from the group consisting of H, (C₁-C₆)alkyl, (C₃-C₆)cycloalkyl, (C₃-C₆)cycloalkyl(C₁-C₆)alkyl, and aryl(C₁-C₆); or R^{m} and Rⁿ taken together with the nitrogen to which they are attached form an aziridino, azetidino, morpholino, piperazino, pyrrolidino or piperidino, which aziridino, azetidino, morpholino, piperazino, pyrrolidino or piperidino is optionally substituted with one or more groups independently selected from the group consisting (C₁-C₆)alkyl;
R^{p} is a detectable group;
each R^{r} and R^{s} is independently selected from the group consisting of H, (C₁-C₆)alkyl, (C₃-C₆)cycloalkyl, (C₃-C₆)cycloalkyl(C₁-C₆)alkyl, and aryl(C₁-C₆); or R^{r} and R^{s} taken together with the nitrogen to which they are attached form an aziridino, azetidino, morpholino, piperazino, pyrrolidino or piperidino, which aziridino, azetidino, morpholino, piperazino, pyrrolidino or piperidino is optionally substituted with one or more groups independently selected from the group consisting (C₁-C₆)alkyl;
X is C═O, C═S, or —CH₂—;
Y is —CH₂— or —NH—;
R^{w} is H or (C₁-C₆)alkanoyl;
R^{y} is H or (C₁-C₆)alkyl; and
R^{z} is H or (C₁-C₆)alkyl.

The invention also provides a pharmaceutical composition comprising a compound of Formula (I) or a pharmaceutically acceptable salt thereof, and a pharmaceutically acceptable excipient.

The invention also provides a method for treating a neurodegenerative disease, a liver disease, or cognitive dysfunction in an animal (e.g., a mammal such as a human) comprising administering a compound of Formula (I) or a pharmaceutically acceptable salt thereof to the animal.

The invention also provides a compound of Formula (I) or a pharmaceutically acceptable salt thereof for use in medical therapy.

The invention also provides a compound of Formula (I) or a pharmaceutically acceptable salt thereof for the prophylactic or therapeutic treatment of a neurodegenerative disease, a liver disease, or cognitive dysfunction.

The invention also provides the use of a compound of Formula (I) or a pharmaceutically acceptable salt thereof to prepare a medicament for treating a neurodegenerative disease, a liver disease, or cognitive dysfunction in an animal (e.g. a mammal such as a human).

The invention also provides processes and intermediates disclosed herein that are useful for preparing a compound of Formula (I) or a salt thereof.

### BRIEF DESCRIPTION OF THE DRAWINGS

FIG. 1. Casp2-selective inhibitors block enzyme-catalyzed Δtau314 production in vitro A) Mass spectrometry (MS) analysis confirmed the identity of a ˜37-kDa tau cleavage product as Δtau314. A representative Western blot (WB) of Casp2-cleaved tau proteins immunoprecipitated by the Δtau314-specific 4F3 antibody was probed using biotin-conjugated pan-tau antibody tau-5 (tau-5-biotin). Liquid chromatography-MS/MS analysis identified the presence of Δtau314 in the ˜37 kDa product as indicated by a representative MS spectrum of its trypsinized signature peptide at the C-terminus. B) Hybrid compounds inhibited Casp2-mediated Δtau314 production with different efficacies. Upper panel, a representative immunoprecipitation (IP, 4F3)/Western blotting (WB, tau-5-biotin) showing Δtau314 in the presence of various hybrid compounds at 10 μM. Δtau314 produced in the absence of any compound (No cmpd.) and a reaction in the absence of enzyme (No Casp2) served as positive and negative controls, respectively. Lower panel, quantification. The level of Δtau314 produced in the absence of any tested compound was defined as 100% (dotted line). At 10 μM, compounds 1 and 30b inhibited tau cleavage at D314 by 96% and 68%, respectively, whereas compound 30a showed no inhibition; compound 1 inhibited Δtau314 production more efficiently than compound 30b. Experiments were repeated six times. Individual values (open circles), means (histograms), and standard deviations (SDs, error bars) are shown. A single-sample t-test was performed to compare the effect of each compound to that of no compound (####, p<0.0001). One-way ANOVA was performed to compare effects of tested compounds (F_{(2, 15)}=48.50, P<0.0001), followed by Tukey's post hoc test (*, p<0.05; ****, p<0.0001). C) Determining the IC₅₀ of compound 1. Upper panel, a representative IP (4F3)/WB (tau-5-biotin) showing Δtau314 in the presence of various concentrations of compound 1. Lower panel, levels of Δtau314 (normalized to the no-compound control) and compound concentrations were fitted to a dose-response curve; the IC₅₀ of compound 1 was determined to be 2.02±0.19 μM. Experiments were performed in duplicate. Means (open circles) and SDs (error bars) are shown.

FIG. 2. Compound 1 blocks the hyper-accumulation of tau caused by the P301S mutation in dendritic spines. A) Untreated neurons co-expressing DsRed and GFP-tagged wild-type tau (top row) were compared with neurons co-expressing DsRed and GFP-tagged P301S tau in the absence (middle row) or presence of compound 1 (bottom row). Arrows denote dendritic spines containing tau. Triangles denote spines devoid of tau. B) Comparison of the average percentage of dendritic spines labeled by DsRed containing GFP-tagged tau between the three groups. n=8 neurons in each group; ANOVA, ***, p<0.001. C) Comparison of the density of dendritic spines (the number of DsRed-labelled dendritic spines/100 μm length of dendrites) between the three groups. n=8 neurons in each group; ANOVA, NS (not significant), P>0.05.

FIG. 3. Treatment with 1 rescue synaptic dysfunction caused by the P301S mutation. A. Representative traces of mEPSCs recorded in Tau−/+ and Tau−/− neurons that have been treated with 30a, 30b, and 1. B and C. Comparisons between the means of mEPSC amplitude (B) or frequency (C) between the above six groups. n=31 neurons in Compound 1 treated groups (−/− and −/+); n=41 in Compound 30b treated groups; n=39 in Compound 30a treated groups; ANOVA, *, p<0.05; **, p<0.01. D. Comparisons of cumulative curves of mEPSC amplitudes recorded in the above six groups. E. A comparison between the rescuing efficacy between 1 and 30b in Tau−/+ neurons. For D and E: Mann-Whitney U Test, *, p<0.05; ***, p, 0.001. For B-E, orange circles, blue circles, and blue triangles denote treatment with 30a, 30b, and 1, respectively. Closed shapes denote Tau−/+ mice and open shapes denote Tau−/− mice.

### DETAILED DESCRIPTION OF THE INVENTION

The following definitions are used, unless otherwise described: halo or halogen is fluoro, chloro, bromo, or iodo. Alkyl, alkoxy, alkenyl, alkynyl, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₈)alkyl, (C₂-C₈)alkyl, C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and and higher homologs and isomers.

The term “alkenyl” refers to an unsaturated alkyl radical having one or more double bonds. Examples of such unsaturated alkyl groups include vinyl, 2-propenyl, crotyl, 2-isopentenyl, 2-(butadienyl), 2,4-pentadienyl, 3-(1,4-pentadienyl) and the higher homologs and isomers.

The term “alkynyl” refers to an unsaturated alkyl radical having one or more triple bonds. Examples of such unsaturated alkyl groups ethynyl, 1- and 3-propynyl, 3-butynyl, and higher homologs and isomers.

The term “alkoxy” refers to an alkyl groups attached to the remainder of the molecule via an oxygen atom (“oxy”).

The term “cycloalkyl” refers to a saturated or partially unsaturated (non-aromatic) all carbon ring having 3 to 8 carbon atoms (i.e., (C₃-C₈)carbocycle). The term also includes multiple condensed, saturated all carbon ring systems (e.g., ring systems comprising 2, 3 or 4 carbocyclic rings). Accordingly, carbocycle includes multicyclic carbocycles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocycles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “alkoxycarbonyl” as used herein refers to a group (alkyl)-O—C(═O)—, wherein the term alkyl has the meaning defined herein.

The term “alkanoyloxy” as used herein refers to a group (alkyl)-C(═O)—O—, wherein the term alkyl has the meaning defined herein.

As used herein, the term “heteroatom” is meant to include oxygen (O), nitrogen (N), sulfur (S) and silicon (Si).

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein a wavy line “ [symbol] ” that intersects a bond in a chemical structure indicates the point of attachment of the bond that the wavy bond intersects in the chemical structure to the remainder of a molecule.

The term “neurodegenerative disease” includes Alzheimer's disease (AD), Huntington's disease (HD), frontotemporal dementia (FTD), Parkinson's disease (PD), excitotoxicity, neuro-ophthalmologic conditions, non-arteritic anterior ischemic optic neuropathy (NAION) and neuroblastoma.

The term “liver disease” includes nonalcoholic steatohepatitis (NASH), Nonalcoholic fatty liver disease (NAFLD), and Nonalcoholic fatty liver.

The term “cognitive dysfunction” includes memory loss as well as conditions associated with stroke.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “mammal” as used herein refers to humans, higher non-human primates, rodents, domestic, cows, horses, pigs, sheep, dogs and cats. In one embodiment, the mammal is a human. The term “patient” as used herein refers to any animal including mammals. In one embodiment, the patient is a mammalian patient In one embodiment, the patient is a human patient.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with -CD₃.

The pharmaceutical compositions of the invention can comprise one or more excipients. When used in combination with the pharmaceutical compositions of the invention the term “excipients” refers generally to an additional ingredient that is combined with the compound of Formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

Specific values listed below for radicals, substituents, and ranges, are for illustration only; they do not exclude other defined values or other values within defined ranges for the radicals and substituents. It is to be understood that two or more values may be combined. It is also to be understood that the values listed herein below (or subsets thereof) can be excluded.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; and aryl can be phenyl, indenyl, or naphthyl.

A specific compound of Formula (I) or a salt thereof is a compound of formula (Ia):

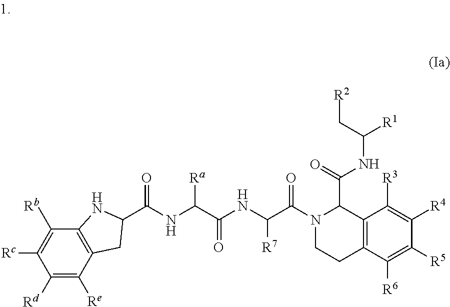

or a salt thereof, wherein:
R¹ is —B(OH)₂, cyano, (C₁-C₆)alkyl, (C₂-C₆)alkenyl, or (C₂-C₆)alkynyl, wherein each (C₁-C₆)alkyl, (C₂-C₆)alkenyl, and (C₂-C₆)alkynyl is optionally substituted with one or more groups independently selected from the group consisting of halo, cyano, oxo, NR^{r}R^{s}, and aryl, wherein each aryl is optionally substituted with one or more groups independently selected from the group consisting of halo, hydroxy, (C₁-C₆)alkyl, cyano, (C₁-C₆)alkoxy, (C₁-C₆)alkoxycarbonyl, carboxy, and (C₁-C₆)alkanoyloxy, and wherein one or more sp3 carbon atoms of each (C₁-C₆)alkyl, (C₂-C₆)alkenyl, and (C₂-C₆)alkynyl is optionally replaced with O, S, S(O), S(O)₂, or NH; or R¹ is —C(═O)R^{p};
R² is —C(═O)OR^{f} or —C(═O)NR^{g}R^{h};
R³ is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R⁴ is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R⁵ is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R⁶ is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R⁷ is H or (C₁-C₆)alkyl that is optionally substituted with (C₁-C₆)alkoxy or (aryl)(C₁-C₆)alkoxy;
R^{a} is H or (C₁-C₆)alkyl that is substituted with —C(═O)OR^{k} or —C(═O)NR^{m}Rⁿ;
R^{b} is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy
R^{c} is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy
R^{d} is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy; R^{e} is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy
R^{f} is selected from the group consisting of H, (C₁-C₆)alkyl, (C₃-C₆)cycloalkyl, (C₃-C₆)cycloalkyl(C₁-C₆)alkyl, and aryl(C₁-C₆);
each R^{g} and R^{h} is independently selected from the group consisting of H, (C₁-C₆)alkyl, (C₃-C₆)cycloalkyl, (C₃-C₆)cycloalkyl(C₁-C₆)alkyl, and aryl(C₁-C₆); or R^{g} and R^{h} taken together with the nitrogen to which they are attached form an aziridino, azetidino, morpholino, piperazino, pyrrolidino or piperidino, which aziridino, azetidino, morpholino, piperazino, pyrrolidino or piperidino is optionally substituted with one or more groups independently selected from the group consisting (C₁-C₆)alkyl;
R^{k} is selected from the group consisting of H, (C₁-C₆)alkyl, (C₃-C₆)cycloalkyl, (C₃-C₆)cycloalkyl(C₁-C₆)alkyl, and aryl(C₁-C₆);
each R^{m} and Rⁿ is independently selected from the group consisting of H, (C₁-C₆)alkyl, (C₃-C₆)cycloalkyl, (C₃-C₆)cycloalkyl(C₁-C₆)alkyl, and aryl(C₁-C₆); or R^{m} and Rⁿ taken together with the nitrogen to which they are attached form an aziridino, azetidino, morpholino, piperazino, pyrrolidino or piperidino, which aziridino, azetidino, morpholino, piperazino, pyrrolidino or piperidino is optionally substituted with one or more groups independently selected from the group consisting (C₁-C₆)alkyl;
R^{p} is a detectable group; and
each R^{r} and R^{s} is independently selected from the group consisting of H, (C₁-C₆)alkyl, (C₃-C₆)cycloalkyl, (C₃-C₆)cycloalkyl(C₁-C₆)alkyl, and aryl(C₁-C₆); or R^{r} and R^{s} taken together with the nitrogen to which they are attached form an aziridino, azetidino, morpholino, piperazino, pyrrolidino or piperidino, which aziridino, azetidino, morpholino, piperazino, pyrrolidino or piperidino is optionally substituted with one or more groups independently selected from the group consisting (C₁-C₆)alkyl.

A specific compound of Formula (I) or a salt thereof is a compound of formula (Ib):

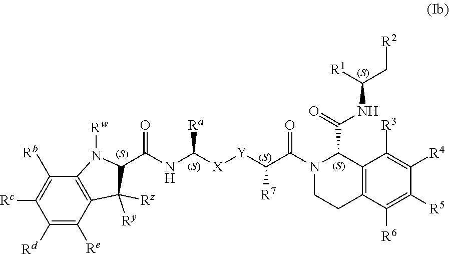

or a salt thereof.

A specific compound of Formula (I) or a salt thereof is a compound of formula (Ic):

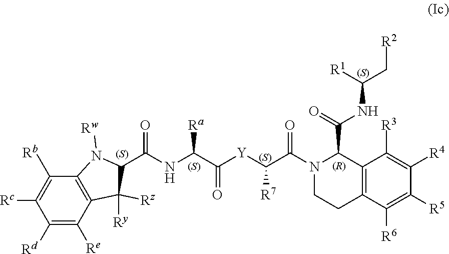

or a salt thereof.

A specific value for R¹ is selected from the group consisting of —CHO, cyano, —C(═O)CH₂O-aryl, —C(═O)CH₂S—CH₂-aryl, —C(═O)CH₂O—C(═O)-aryl, —B(OH)₂,

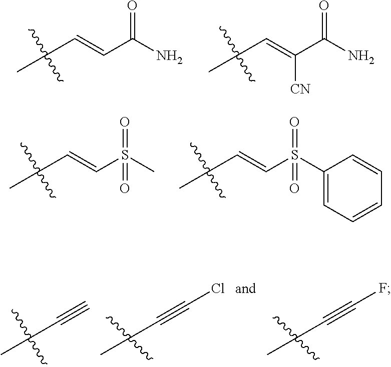

wherein any aryl is optionally substituted with 1, 2, 3, or 4 halo.

A specific value for R¹ is —CHO.

A specific value for R¹ is cyano.

A specific value for R² is —C(═O)OR^{f}; wherein R^{f} is H, methyl, ethyl, or isopropyl.

A specific value for R² is —C(═O)OH.

A specific value for R² is —C(═O)NR^{g}R^{h}; wherein R⁹ is H and R^{h} is H or methyl.

A specific value for R³ is H, methyl, ethyl, propyl, isopropyl, F, Cl, cyano, or hydroxy.

A specific value for R⁴ is H, methyl, ethyl, propyl, isopropyl, F, Cl, cyano, or hydroxy.

A specific value for R⁵ is H, methyl, ethyl, propyl, isopropyl, F, Cl, cyano, or hydroxy.

A specific value for R⁶ is H, methyl, ethyl, propyl, isopropyl, F, Cl, cyano, or hydroxy.

A specific value for R⁷ is methyl or tert-butyl.

A specific value for R^{a} is 4-carboxypropyl.

A specific value for R^{a} is propyl that is substituted at the 3-position with —C(═O)OR^{f};

wherein R^{f} is H, methyl, ethyl, or isopropyl.

A specific value for R^{a} is propyl that is substituted at the 3-position with —C(═O)NR^{g}R^{h}; wherein R^{g} is H and R^{h} is H or methyl.

A specific value for R^{b} is H, F, Cl, cyano, or hydroxy.

A specific value for R^{c} is H, F, Cl, cyano, or hydroxy.

A specific value for R^{d} is H, F, Cl, cyano, or hydroxy.

A specific value for R^{e} is H, F, Cl, cyano, or hydroxy.

A specific value for R¹ is —C(═O)R^{p} wherein R^{p} is a detectable group selected from the group consisting of:

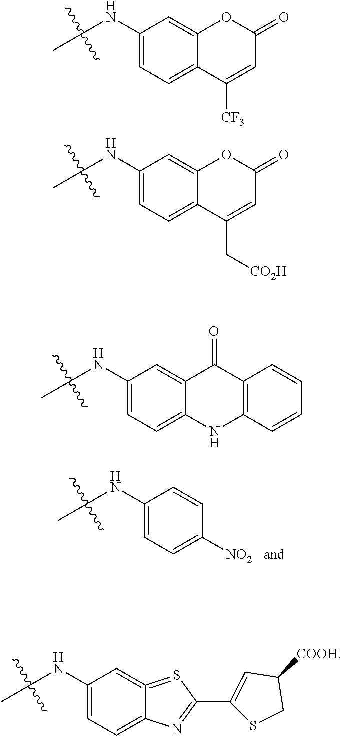

A specific compound of Formula (I) or a salt thereof is:

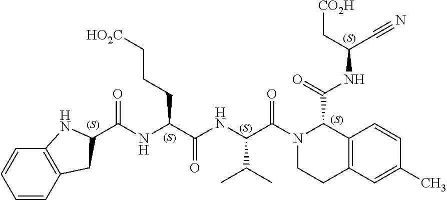

or a salt thereof.

A specific compound of Formula (I) or a salt thereof is selected from the group consisting of:

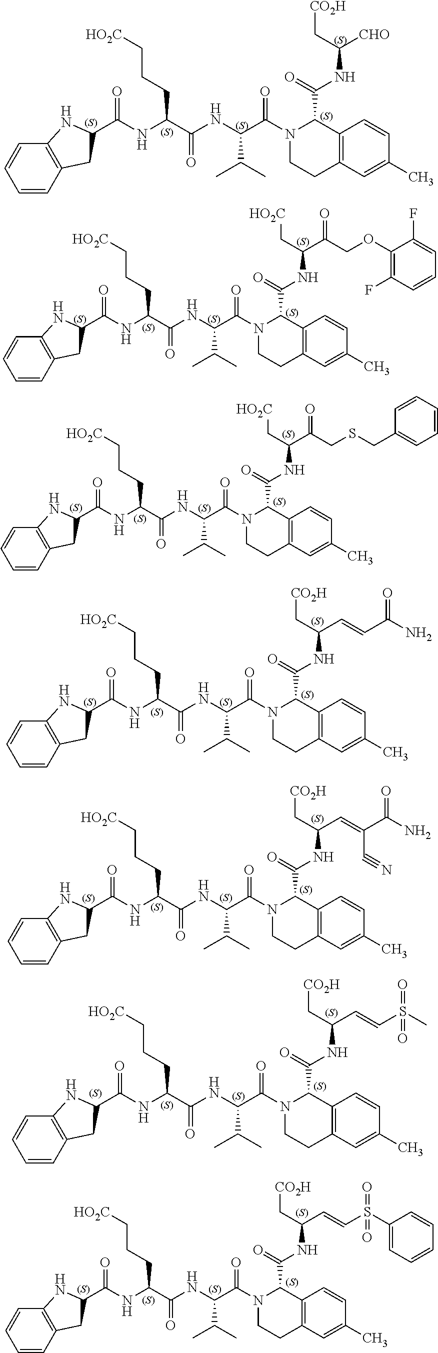

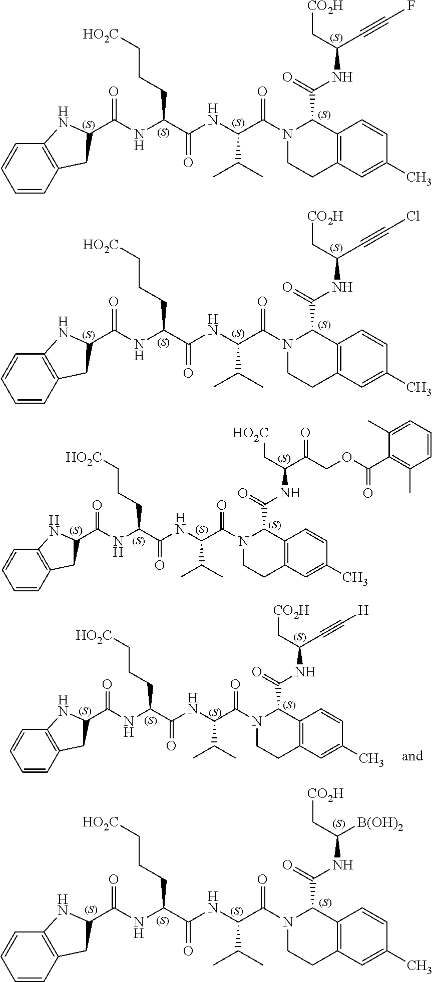

and salts thereof.

A specific compound of Formula (I) or a salt thereof is a compound of formula (Id):

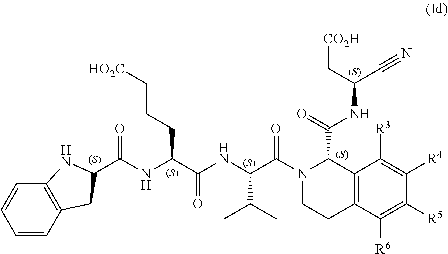

or a salt thereof, wherein:
R³ is H, methyl, ethyl, propyl, isopropyl, F, Cl, CN, or hydroxy;
R⁴ is H, methyl, ethyl, propyl, isopropyl, F, Cl, CN, or hydroxy;
   R⁵ is H, methyl, ethyl, propyl, isopropyl, F, Cl, CN, or hydroxy; and
   R⁶ is H, methyl, ethyl, propyl, isopropyl, F, Cl, CN, or hydroxy.
   A specific compound of Formula (I) or a salt thereof is selected from the group consisting of:

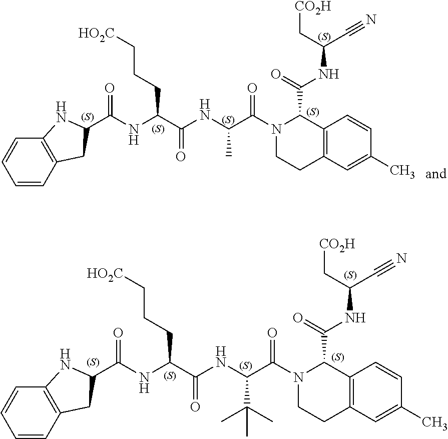

and salts thereof.

A specific compound of Formula (I) or a salt thereof is selected from the group consisting of:

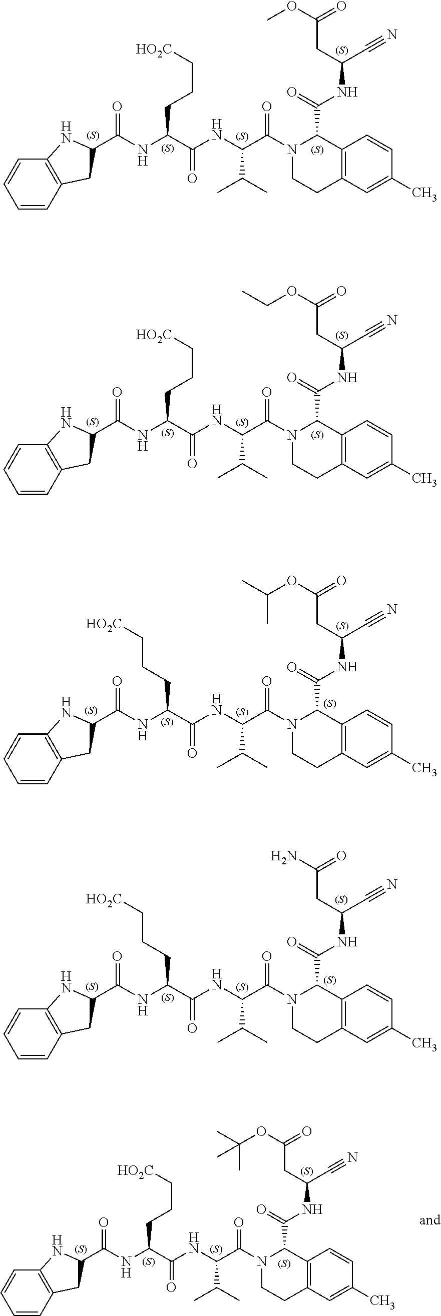

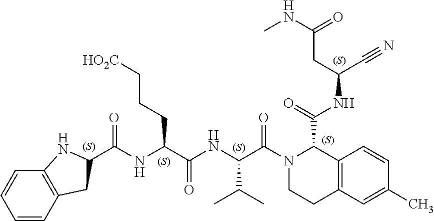

and salts thereof.

A specific compound of Formula (I) or a salt thereof is selected from the group consisting of:

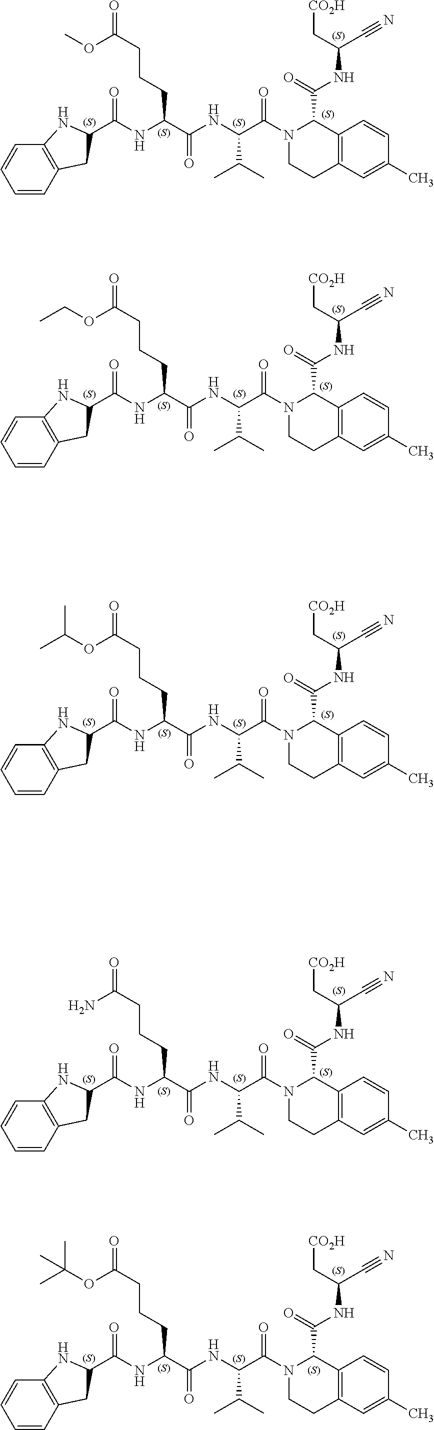

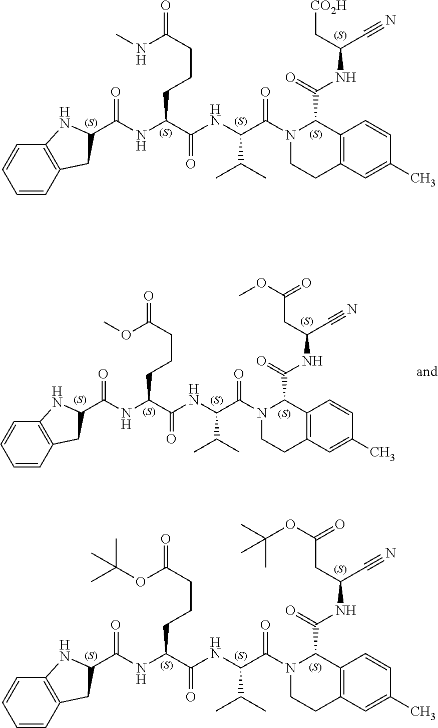

and salts thereof.

A specific compound of Formula (I) or a salt thereof is a compound wherein: R^{b} is H, F, Cl, CN, or hydroxy; R^{c} is H, F, Cl, CN, or hydroxy; R^{d} is H, F, Cl, CN, or hydroxy; and R^{e} is H, F, Cl, CN, or hydroxy.

A specific compound of Formula (I) or a salt thereof is selected from the group consisting of:

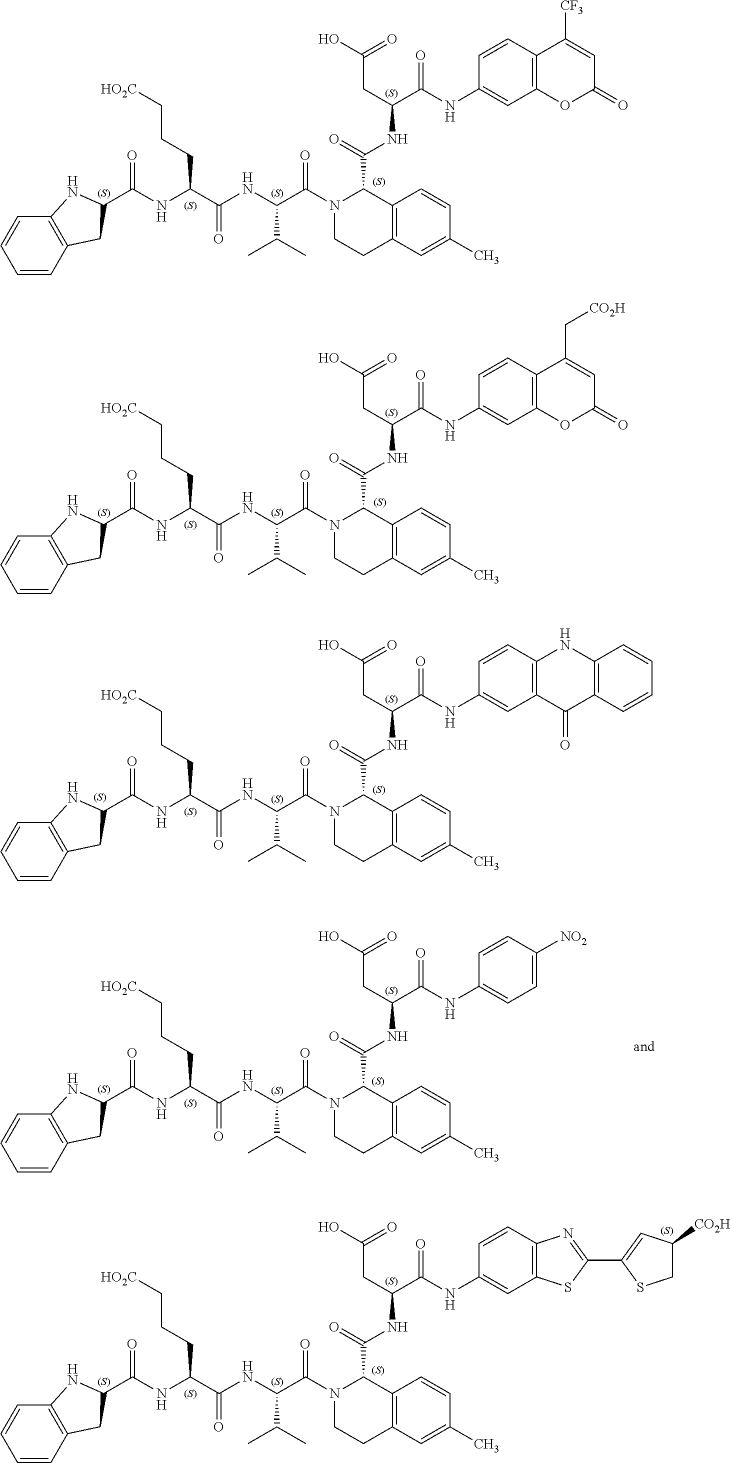

and salts thereof.

A specific compound or salt is a compound selected from the group consisting of:

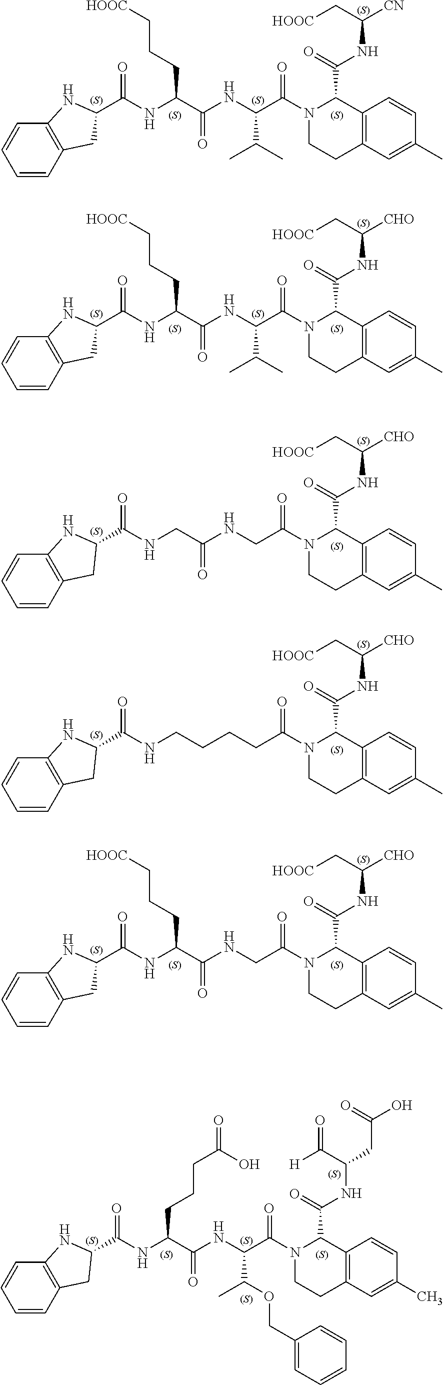

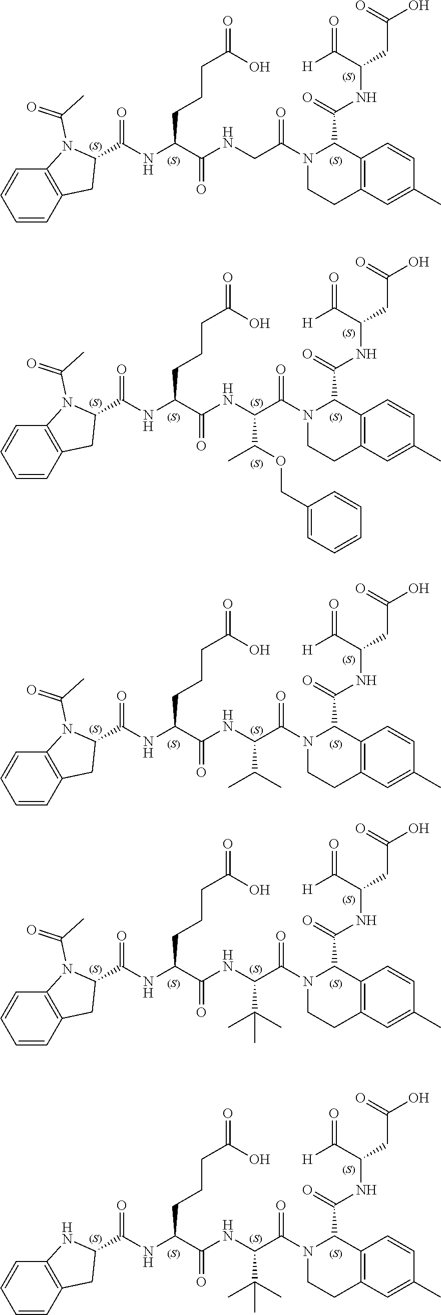

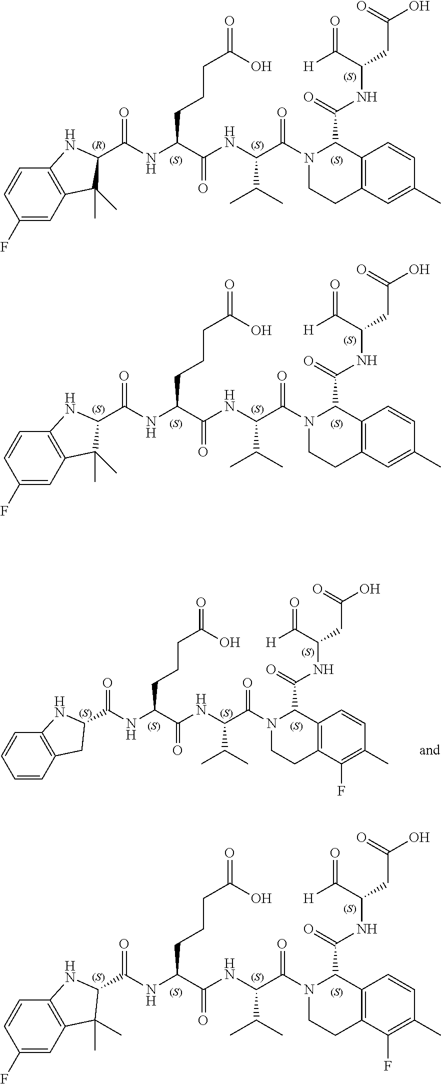

or a salt thereof.

A specific value for R^{w} is acyl.

A specific value for R^{w} is H.

A specific value for R^{y} is H.

A specific value for R^{y} is methyl.

A specific value for R^{z} is H.

A specific value for R^{z} is methyl.

A specific value for X is —C(═O)—.

A specific value for X is —C(═S)—.

A specific value for X is —CH₂—.

A specific value for Y is —CH₂.

A specific value for Y is —NH—.

Processes for preparing compounds of Formula (I) are provided as further embodiments of the invention and are illustrated by the following procedures in which the meanings of the generic radicals are as given above unless otherwise qualified.

A compound of Formula (I) can be prepared as illustrated in the following Scheme by coupling an acid of formula 101 with an amine of formula 102.

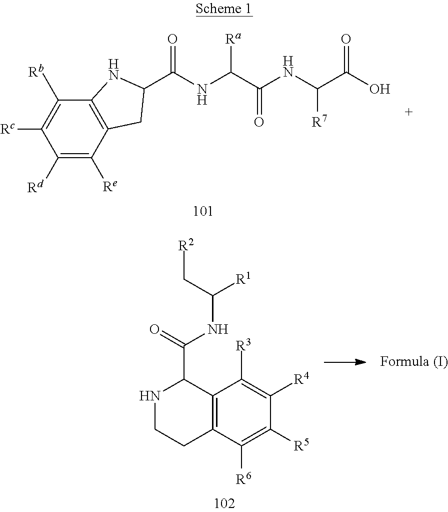

In cases where compounds are sufficiently basic or acidic, a salt of a compound of Formula (I) can be useful as an intermediate for isolating or purifying a compound of Formula (I). Additionally, administration of a compound of Formula (I) as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of Formula (I) can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of Formula (I) to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of Formula (I) can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents. Accordingly, in one embodiment the invention also provides a composition comprising a compound of Formula (I), or a pharmaceutically acceptable salt thereof, at least one other therapeutic agent, and a pharmaceutically acceptable diluent or carrier. The invention also provides a kit comprising a compound of Formula (I), or a pharmaceutically acceptable salt thereof, at least one other therapeutic agent, packaging material, and instructions for administering the compound of Formula (I) or the pharmaceutically acceptable salt thereof and the other therapeutic agent or agents to an animal to treat cognitive dysfunction.

The invention will now be illustrated by the following non-limiting Examples.

### EXAMPLES

### General

All solution phase experiments were carried out under nitrogen in oven-dried glassware. All starting materials, solvents, and reagents were purchased from commercial sources and used without further purification. Aspartic acid-derived semi-carbazide Merrifield resin was prepared by using the previously reported procedure (WO2001/027085). Intermediates were purified by flash chromatography using 230-400 mesh silica gel. ¹H NMR spectra were recorded on Bruker 400 MHz spectrometer in deuterated solvents. Final compounds for the biological assays were purified by preparative HPLC. Chemical shifts are reported in parts per million (ppm).

Final compounds 1 and 3 were purified by preparative-HPLC, utilizing a Waters Atlantis Zoraba×SB C18 column, gradient 0-100% B (A=water, B=acetonitrile) over 18 minutes, injection volume 1 mL, flow=20 ml/min. UV spectra were recorded at 215 nm using a Gilson 119 UV/vis detector system. LCMS purity was determined on Acquity UPLC (Waters Corporation) equipped with an Acquity BEH UPLC C18 column (2.1×50 mm, 1.7 μm) for separation. Compound absorbance was detected at 214 nm using a photo diode array detector. Mass data was acquired using a Micromass ZQ mass spectrometer.

LC was completed using a gradient method with each of the following mobile phase systems: Mobile Phase A2: 95% Water, 5% Acetonitrile, 0.1% Formic Acid; Mobile Phase B2: 95% Acetonitrile, 5% Water, 0.1% Formic Acid; Mobile Phase A1: 95% Water, 5% Methanol, 0.1% Formic Acid; Mobile Phase B1: 95% Methanol, 5% Water, 0.1% Formic Acid.

Analytical HPLC-MS was performed on Shimadzu LCMS-2010 EV systems using reverse phase Atlantis C18 columns, gradient 5-100% B (A=water/0.1% formic acid, B=acetonitrile/0.1% formic acid) over 6 min, flow=0.6 mL/min.

Mass spectra were obtained over the range m/z 150-850 at a sampling rate of 2 scans per second using Waters LCT or analytical HPLC-MS systems using reverse phase Water Atlantis C18 columns gradient 5-100% B (A=water/0.1% formic acid, B=acetonitrile/0.1% formic acid).

For the compounds 30a and 30b, preparative HPLC was performed on a Knauer system (consisting of two K-1800 pumps, a K-2001 detector) using a Phenomenex Gemini C18 column (250×21 mm, 5 μm) at a flow rate of 15 mL/min. As a mobile phase, mixtures of MeCN and 0.1% TFA in water were used. UV detection was carried out at 220 nm. Purity analysis of the compounds 30a and 30b was performed on an Agilent 1100 HPLC system (equipped with an Instant Pilot controller, a G1312A binary pump, a G1329A ALS autosampler, a G1379A vacuum degasser, a G1316A column compartment, and a G1315B diode array detector) using a Phenomenex Kinetex XB-C18 column (250×4.6 mm, 5 μm) at 30° C. oven temperature using mixtures of MeCN and 0.1% TFA in water as mobile phase. Absorbance was detected at 220 nm. Purity control for 30a and 30b was performed as recently reported by Bresinsky et al., 2022, ACS Pharmacol Transl Sci.; published online. //doi.org/10.1021/acsptsci.1c00251.

Racemic 6-methyl-tetrahydroisoquinoline-1-carboxylic acid (6-Me-1-THIQCOOH) was obtained from a commercial source and was outsourced for chiral resolution by supercritical fluid chromatography (SFC).

Compounds 30a, 30b, and 3 were prepared via solid-phase peptide synthesis using aspartic acid modified semi-carbazide resin (2) as described by Maillard et al., 2011, Bioorganic & Medicinal Chemistry 19(19), 5833-51.

### Example 1. Synthesis of (8)-6-(((S)-1-((8)-1-(((S)-2-carboxy-1-cyanoethyl)carbamoyl)-6-methyl-3,4-dihydroisoquinolin-2(1H)-yl)-3-methyl-1-oxobutan-2-yl)amino)-5-((S)-indoline-2-carboxamido)-6-oxohexanoic acid (1)

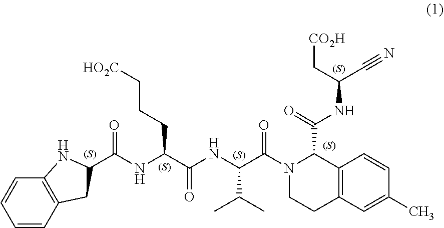

To a solution of tripeptide carboxylic acid 18 (267 mg, 0.39 mmol) in 5 mL of acetonitrile were added HATU (102 mg, 0.39), 2,4,6-collidine (1.56 mmol, 0.21 mL), and 20 (100 mg, 0.26 mmol). The reaction mixture was stirred at rt. for 6 h, and the solvent was evaporated. The crude residue was diluted with water (20 mL) and extracted with ethyl acetate (4×20 mL). The combined organic layer was sequentially washed with sat. aq. NaHCO₃ (20 mL), ice-cold 10% aq. KHSO₄ (20 mL), and brine (20 mL). The organic layer was collected over anhyd. Na₂SO₄, filtered, and concentrated under reduced pressure to obtain the crude peptide. The crude residue was dissolved in a minimum volume of dichloromethane (3 mL) and transferred to vigorously stirring phosphoric acid (85%, 3 mL). The completion of the reaction was monitored by UPLC-MS. The reaction mixture was diluted with water (5 mL) and extracted with ethyl acetate (4×10 mL). The organic layer was dried over anhyd. Na₂SO₄, filtered, and concentrated to obtain the crude carboxylic acid.

The crude product was dissolved in acetonitrile (10 mL), and diethyl amine (3 mL) was added to it. The reaction mixture was stirred at rt. for 3 h and was concentrated under reduced pressure. The crude residue was triturated sequentially with hexane, diethyl ether, and the solvent decanted. The residue was dried under reduced pressure and dissolved in dimethylformamide (DMF, 3 mL). Palladium (10%, 10 mg) was added, and the reaction mixture was purged with hydrogen gas for 5 min and stirred under hydrogen (1 atm). The completion of the reaction was monitored by UPLC-MS. The reaction mixture was filtered over a thin layer of celite. The celite layer was washed with 2 mL of DMF. The solvent was evaporated, and the residue was purified by preparative HPLC. Lyophilization of the pure fractions provided the product 1 as a colorless amorphous powder (38 mg): RP-HPLC: 99%. ¹H NMR (400 MHz, CD₃OD) δ 9.02-8.72 (m, 0.5H), 8.43 (m, 0.7H), 8.13 (m, 1H), 7.64-7.29 (m, 1H), 7.25-6.43 (m, 7H), 5.58 (s, 0.6H), 5.40 (s, 0.4H), 4.63-4.15 (m, 3H), 4.07-3.83 (m, 1H), 3.81-3.61 (m, 1H), 3.60-3.3 (m, 1H), 3.13-2.45 (m, 5H), 2.38-1.96 (m, 5H), 1.95-1.08 (m, 5H), 0.99-0.67 (m, 5H), 0.55 (d, J=6.8 Hz 1H). HRMS (ESI) m/z [M+H⁺] calculated for C₃₅H₄₃N₆O₈⁺: 675.3137, found 675.3145.

The intermediate tri-peptide carboxylic acid 18 and compound (20) were prepared as follows.

### a. Synthesis of ((S)-2-((S)-1-(((9H-fluoren-9-yl)methoxy)carbonyl)indoline-2-carboxamido)-6-(tert-butoxy)-6-oxohexanoyl)-L-valine (18)

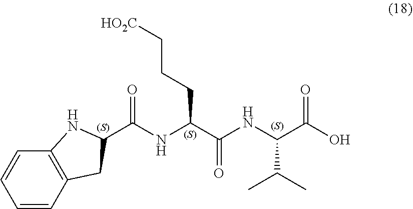

Step 1. Loading of Fmoc-Val-OH on resin—To 1 g of 2-chlorotrityl chloride resin in a polyprep column 50 mL of CH₂C₁₂ was added. After 0.5 hours the solvent was pushed out from the column under nitrogen flow. A solution of Fmoc-Val-OH (225 mg, 0.66 mmol) and 2,4,6-collidine (1.0 mL, 7.56 mmol) in 25 mL of CH₂C₁₂ was transferred to the resin. The mixture was rocked for 3 hours at room temperature. The solvent was pushed out under nitrogen flow and the resin was washed with CH₂C₁₂ (3×50 mL). Then the resin was treated with a capping solution of CH₂C₁₂:MeOH:DIPEA (40 mL:4 mL:2 mL) for 1 hour and the solvent was pushed out under nitrogen flow. The resin was washed with CH₂C₁₂ (3×50 mL) and DMF (3×50 mL).

Step 2. Fmoc deprotection—The resin from step 1 was treated with piperidine (30 mL, 20% in DMF) for 0.5 hours. The solvent was pushed out under nitrogen flow and the resin was washed with DMF (3×50 mL). Step 3. Coupling of Fmoc-hGlu-OH— A solution of Fmoc-hGlu-OH (308 mg, 0.70 mmol), HATU (266 mg, 0.70 mmol), and collidine (0.20 mL, 1.5 mmol) in CH₂C₁₂ (25 mL) was added. The reaction was rocked for 3 hours at rt. The solvent was pushed out under nitrogen flow and Fmoc deprotection was carried out as described in Step 2. Fmoc-Idc-OH was coupled as described in Step 3 to complete the tripeptide sequence. The resin was sequentially washed with DMF, CH₂C₁₂, DMF, CH₂C₁₂ (3×50 mL). The peptide was cleaved from the resin by treating with 20% HFIP/CH₂C₁₂ solution for 2 hours. The peptide solution was collected under nitrogen flow, evaporated, and subjected to purification by preparative HPLC. The product (18) was obtained as an amorphous powder (210 mg, 0.307 mmol) after lyophilization of the pure fractions. MS (ESI) m/z [M+H⁺] 684.4. ¹H NMR (400 MHz, CDCl₃) δ 7.62 (dd, J 7.6, 3.9 Hz, 2H), 7.45 (t, J 8.4 Hz, 2H), 7.25 (m, 2H), 7.21-7.08 (m, 4H), 6.93 (d, J 7.4 Hz, 1H), 6.78 (m, 1H), 5.61 (m, 2H), 4.75 (dd, J=11.1, 4.3 Hz, 1H), 4.65-4.01 (m, 4H), 3.54-3.13 (m, 1H), 3.02 (d, J=16.1 Hz, 1H), 2.00 (m, 2H), 1.60 (m, 1H), 1.22 (s, 9H), 1.15-1.09 (m, 1H), 0.80-0.69 (m, 6H).

### b. Synthesis of Benzyl(S)-3-cyano-3-((S)-6-methyl-1,2,3,4-tetrahydroisoquinoline-1-carboxamido)propanoate (20)

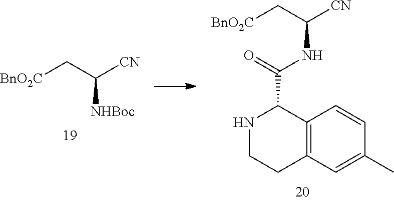

To a vigorously stirring phosphoric acid (5 mL, 85%), a solution of compound 19 (402 mg, 1.32 mmol) in dichloromethane (2 mL) was added dropwise at room temperature. After 3 hours stirring at room temperature, the reaction mixture was cooled to 0° C. in ice-bath and neutralized using sat. aq. NaHCO₃ solution. The resulting aqueous mixture was extracted with ethyl acetate (20 mL×4) and collected over anhydrous. Na₂SO₄. Filtration followed by concentration under reduced pressure gave the unprotected amine that was utilized for the next step without further purification.

The compound S-17 (275 mg, 0.66 mmol), HATU (250 mg, 0.66 mmol), and 2,4,6 collidine (0.26 mL, 1.98 mmol) in anhydrous acetonitrile (10 mL) at 0° C. was added a solution of the unprotected amine amine (in 5 mL acetonitrile) prepared above and the reaction mixture was stirred at room temperature for 4 hours. The reaction was concentrated under reduced pressure, diluted with water (20 mL), and extracted with ethyl acetate (4×20 mL). The combined organic layer was sequentially washed with sat. aq. NaHCO₃ (20 mL), ice-cold 10% aq. KHSO₄ (20 mL), and brine (20 mL). The organic layer was collected over anhydrous sodium sulfate, filtered, and concentrated under reduced pressure to obtain the crude amide. The crude product was dissolved in acetonitrile (10 mL) and diethyl amine (5 mL) was added. The reaction mixture was stirred at room temperature for 3 hours then concentrated under reduced pressure. The crude residue was triturated with hexane and the hexane layer was decanted. The residue was purified by silica gel flash chromatography (eluent 1% aq. NH₃ in 10% MeOH/DCM) to obtain amine 20 as light-yellow solid (110 mg, 44% yield). MS (ESI) m/z [M+H⁺] 378.2. ¹H NMR (400 MHz, CDCl₃) δ 8.39 (d, J=9.1 Hz, 1H), 7.37 (qd, J=7.0, 3.8 Hz, 7H), 7.01 (dd, J=8.0, 1.9 Hz, 1H), 6.91 (s, 1H), 5.21 (dt, J=9.0, 5.3 Hz, 1H), 5.15 (s, 2H), 4.57 (s, 1H), 3.08-2.90 (m, 3H), 2.88 (d, J 5.1 Hz, 1H), 2.80 (d, J 5.7 Hz, 1H), 2.72 (ddd, J=25.5, 13.9, 8.1 Hz, 3H), 2.31 (s, 3H).

The intermediate compound S-6 was prepared as follows.

### c. Synthesis of Compound (S)-17

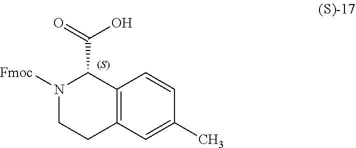

A mixture of enantiopure 6-methyl-1,2,3,4-tetrahydroisoquinoline-1-carboxylic acid 1.23 g, 5.75 mmol), dioxane (30 mL), 10% aqueous (aq.) NaHCO₃ (12 mL), and Fmoc-OSu (2.13 g, 6.32 mmol) was stirred at room temperature for 18 hours. The reaction mixture was cooled to 0° C. and acidified to pH 3 by addition of aq. HCl (1N) and was extracted with ethyl acetate (50 mL×3). The combined organic layer was washed with brine and collected over anhydrous (anhyd.) Na₂SO₄. Filtration followed by evaporation provided the crude residue that was purified by silica gel flash chromatography to obtain the titled compound (S)-6 as viscous mass in 90% yield. MS (ESI) m/z [M+H⁺] 414.0. ¹H NMR (400 MHz, CDCl₃) δ 6.95-7.83 (m, 11H), 5.62 (s, 0.6H), 5.33 (s, 0.4H), 4.38-4.61 (m, 2H), 4.16-4.36 (m, 1H), 3.66-3.97 (m, 2H), 2.73-3.06 (m, 2H), 2.29-2.37 (m, 3H). [α]_{D}^{25.3}−5.32 (c 0.8, DMF).

### Example 2. Synthesis of (S)-6-(((S)-1-((S)-1-(((S)-1-carboxy-3-oxopropan-2-yl)carbamoyl)-6-methyl-3,4-dihydroisoquinolin-2(1H)-yl)-3-methyl-1-oxobutan-2-yl)amino)-5-((S)-indoline-2-carboxamido)-6-oxohexanoic acid hydrotrifluoroacetate (2)

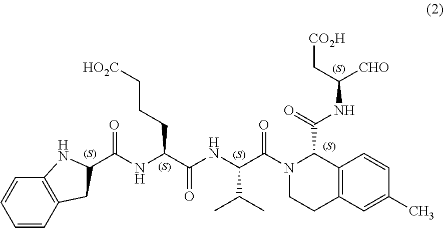

Using a procedure similar to that described in Example 1, but substituting the requisite staring materials, the title compound (2) was prepared as a fluffy white solid (19 mg): RP-HPLC: 97%. ¹H NMR (400 MHz, DMSO-d₆) δ12.1-11.7 (m, 0.6H), 9.06-8.76 (m, 1H), 7.61-6.75 (m, 7H), 6.06-4.20 (m, 4H), 4.15-2.54 (m, 13H), 2.93-1.67 (m, 4H), 3.11-2.57 (m, 4H), 1.43-0.28 (m, 7H). FIRMS (ESI): m/z [M+H⁺] calculated for C₃₅H₄₄N₅O₉⁺: 678.3134, found 678.3133.

### General Procedure for the Synthesis of Examples 3-15 (Compounds 3-15)

Aspartic acid loaded semicarbazide amino-Merrifield resin (300 mg, 0.188 mmol, 1 eq) was weighed into a fritted 10 mL syringe. Subsequently, 5 mL of a mixture of piperidine 20% in DMF was drawn to remove the N-terminal Fmoc protecting group. The syringe was shaken on an orbital shaker at 35° C. for 15 minutes. The orbital shaker was covered with a box, which was insulated from the inside with aluminum foil. An infrared lamp was placed on an aperture on top. To keep the temperature constant at 35° C. the lamp was controlled by a thermostat. The liquid was then removed with the aid of a vacuum flask and the residual resin was washed with DMF (3×8 mL). The coupling of the amino acids to the N-terminus was performed as follows. The corresponding amino acid (5 eq) and HATU (357 mg, 0.94 mmol, 5 eq) were weighed in two separate Erlenmeyer flasks. Subsequently, both were dissolved in 3-4 mL of a mixture of DMF/NMP (8/2 v/v). Then N,N-diisopropylethylamine (164 μL, 0.94 mmol, 5 eq) was added to the solution of HATU. Subsequently, both solutions were drawn into the resin-loaded syringe and shaken at 35° C. for 45 min. The liquid was then removed with the aid of a vacuum flask and the residual resin was washed with DMF (3×8 mL).

The coupling and deprotection steps were repeated until the desired pentapeptide was built up. Then the N-terminal Fmoc protecting group was removed using piperidine in DMF (20%). For selected compounds, the N-terminus of the peptide was acetylated by dissolving acetic anhydride (178 μL, 1.88 mmol, 10 eq) and N,N-diisopropylethylamine (328 μL, 1.88 mmol, 10 eq) in 6-8 mL DMF. The solution was drawn into the syringe and shaken for 30 minutes at room temperature (optional). After completion, the liquid was removed and the resin was washed with DMF (2×8 mL), methanol (2×8 mL), dichloromethane (2×8 mL), and finally with diethyl ether (2×8 mL). The peptide was cleaved off the resin and the side chains were deprotected by drawing 6 mL of trifluoroacetic acid 90% in water. The syringe was shaken for 1 h at room temperature. The liquid was then poured into a round-bottomed flask. The step was repeated and then the cleavage cocktail was diluted with 50 mL water and freeze-dried. The crude product was purified by HPLC, yielding the corresponding peptide.

### Example 3. Synthesis of (S)-3-((S)-2-(((S)-indoline-2-carbonyl)glycylglycyl)-6-methyl-1,2,3,4-tetrahydroisoquinolin-1-carboxamido)-4-oxobutanoic acid hydrotrifluoroacetate (3)

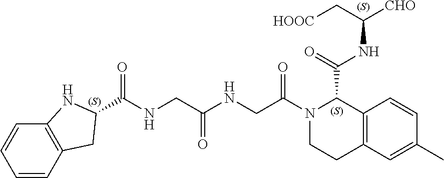

The title compound was synthesized according to the general procedure (using 150 mg resin) yielding a fluffy white solid (22 mg): RP-HPLC: 95%. ¹H NMR (300 MHz, DMSO) c 9.42-9.24 (m, 1H), 886-8.66 (m, 1H), 8.20-8.00 (m, III), 7.49-653 (m, 7H), 5.88-5.15 (m, 2H), 4.62-3.75 (m, 7H), 3.34-3.27 (m, 2H), 3.15-2.57 (m, 6H), 2.26 (s, 6H). HRMS (ESI-MS): m/z [M+H⁺] calculated for C₂₈H₃₂N₅O₇⁺: 550.2296, found 550.2292.

### Example 4. Synthesis of (S)-3-((S)-2-(5-((S)-indoline-2-carboxamido)pentanoyl)-6-methyl-1,2,3,4-tetrahydroisoquinoline-1-carboxamido)-4-oxobutanoic acid hydrotrifluoroacetate (4)

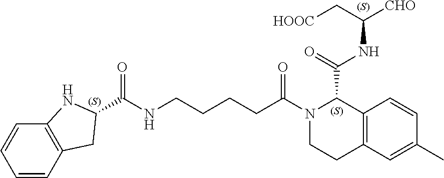

The title compound was synthesized according to the general procedure (using 150 mg resin) yielding a fluffy white solid (17 mg): IMP-HPLC: 95%. ¹H NMR (300 MHz, DMSO) δ 9.30 (d, J=10.6 Hz, 1H), 8.85-8.71 (m, 1H), 8.00-7.52 (m, 1H), 7.46-6.43 (m, 7H), 5.88-5.18 (m, 1H), 4.57-3.60 (m, 2H), 3.32-3.21 (m, 2H), 3.19-2.82 (m, 6H), 2.82-2.61 (m, 2H), 2.46-2.30 (m, 2H), 225 (s, 3H), 1.82-0.98 (in, 3H). FIRMS (ESI-MS): m/z [M+H⁺] calculated for C₂₉H₃₅N₄O₆⁺: 535.2551, found 535.2548.

### Example 5. Synthesis of (S)-6-((2-((S)-1-(((S)-1-carboxy-3-oxopropan-2-yl)carbamoyl)-6-methyl-3,4-dihydroisoquinolin-2(1H)-yl)-2-oxoethyl)amino)-5-((S)-indoline-2-carboxamido)-6-oxohexanoic acid hydrotrifluoroacetate (5)

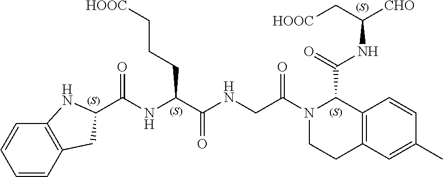

The title compound was synthesized according to the general procedure (using 150 mg resin) yielding a fluffy white solid (34 mg): RP-HPLC: >99%. ¹H NMR (300 MHz, DMSO-d₆) δ 9.46-9.20 (m, 1H), 9.12-8.75 (m, 1H), 8.40-8.18 (m, 1H), 8.00-7.84 (in, H), 7.47-7.24 (m, 1H), 7.20-6.90 (m, 5H), 6.84-6.51 (m, 2H), 5.99-5.21 (m, 2H), 4.50-4.21 (m, 2H), 3.39-3.23 (m, 1H), 3.10-2.65 (m, 4H), 2.38-1.98 (m, 7H), 1.86-1.16 (m, 6H). HRMS (ESI-MS): m/z [M+H⁺] calculated for C₃₂H₃₈N₅O₉⁺: 636.2591, found 636.2665.

### Example 6. Synthesis of (S)-6-(((2S,3S)-3-(benzyloxy)-1-((S)-1-(((S)-1-carboxy-3-oxopropan-2-yl)carbamoyl)-6-methyl-3,4-dihydroisoquinolin-2(1H)-yl)-1-oxobutan-2-yl)amino)-5-((S)-indoline-2-carboxamido)-6-oxohexanoic acid hydrotrifluoroacetate (6)

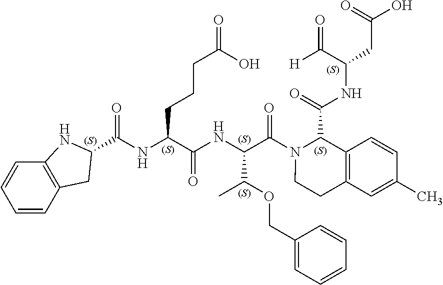

The title compound was synthesized according to the general procedure (using 50 mg resin) yielding a fluffy white solid (21 mg): RP-HPLC: 97%. ¹H NMR (300 MHz, DMSO-d₆) δ 9.27 (s, 1H), 9.02-8.45 (m, 1H), 8.39-8.26 (m, 1H), 7.93-7.79 (m, 1H), 7.39-7.22 (m, 6H), 7.07-6.90 (m, 5H), 5.82-5.36 (m, J=59.4, 53.7 Hz, 1H), 4.60-4.43 (m, J=14.1 Hz, 3H), 4.30-4.16 (m, 1H), 4.01-3.91 (m, 1H), 3.91-3.81 (m, 1H), 3.63-3.51 (m, 2H), 2.97-2.68 (m, 6H), 2.29-2.20 (m, 5H), 1.54-1.34 (m, 4H), 1.16 (d, J=6.2 Hz, 3H). HRMS (ESI-MS): m/z [M+H⁺] calculated for C₄₁H₄₇N₅O₁₀⁺: 669.3323, found 669.3414.

### Example 7. Synthesis of (S)-5-((S)-1-acetylindoline-2-carboxamido)-6-((2-((S)-1-(((S)-1-carboxy-3-oxopropan-2-yl)carbamoyl)-6-methyl-3,4-dihydroisoquinolin-2(1H)-yl)-2-oxoethyl)amino)-6-oxohexanoic acid (7)

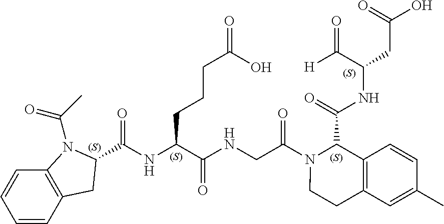

The title compound was synthesized according to the general procedure (using 150 mg resin) yielding a fluffy white solid (19 mg): RP-HPLC: 98%. ¹H NMR (300 MHz, DMSO-d₆) δ 9.30 (d, J=13.7 Hz, 1H), 8.53 (s, 1H), 8.03 (d, J=8.2 Hz, 1H), 7.90 (d, J=8.2 Hz, 1H), 7.20-6.92 (m, 7H), 5.74-5.63 (m, 1H), 5.51-5.44 (m, J=9.1 Hz, 1H), 5.28-5.22 (m, 1H), 5.12-4.98 (m, 1H), 4.10 (s, 2H), 3.03-2.71 (m, 8H), 2.42-2.30 (m, 2H), 2.26 (s, 5H), 1.99 (s, 2H), 1.79-1.64 (m, 2H), 1.58-1.50 (m, 2H). HRMS (ESI-MS): m/z [M−H⁺] calculated for C₃₄H₃₉N₅O₁₀⁻: 677.2697, found 667.2626.

### Example 8. Synthesis of (S)-5-((S)-1-acetylindoline-2-carboxamido)-6-(((2S,3S)-3-(benzyloxy)-1-((S)-1-(((S)-1-carboxy-3-oxopropan-2-yl)carbamoyl)-6-methyl-3,4-dihydroisoquinolin-2(1H)-yl)-1-oxobutan-2-yl)amino)-6-oxohexanoic acid (8)

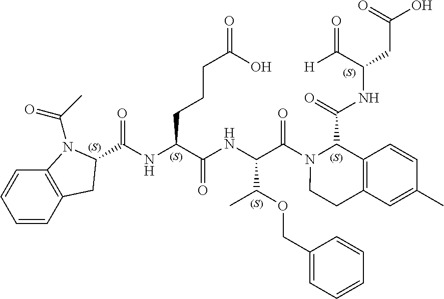

The title compound was synthesized according to the general procedure (using 150 mg resin) yielding a fluffy white solid (24 mg): RP-HPLC: 99%. ¹H NMR (300 MHz, DMSO-d₆) δ 9.27 (d, 1H), 8.50 (s, 1H), 8.04 (d, J=8.4 Hz, 1H), 7.86 (s, 1H), 7.34-7.10 (m, 12H), 5.56 (d, J=5.7 Hz, 1H), 5.09-4.99 (m, 1H), 4.95-4.89 (m, 1H), 4.49 (s, 2H), 3.88-3.82 (m, 1H), 3.56-3.52 (m, 2H), 3.01-2.83 (m, J=11.4 Hz, 6H), 2.27-2.24 (m, 5H), 1.99 (s, 3H), 1.78-1.52 (m, J=39.7 Hz, 4H), 1.13 (d, J=7.0 Hz, 3H). HRMS (ESI-MS): m/z [M−H⁺] calculated for C₄₃H₄₉N₅O₁₁⁻: 811.3429, found 811.3364.

### Example 9. Synthesis of (S)-5-((S)-1-acetylindoline-2-carboxamido)-6-(((S)-1-((S)-1-(((S)-1-carboxy-3-oxopropan-2-yl)carbamoyl)-6-methyl-3,4-dihydroisoquinolin-2(1H)-yl)-3-methyl-1-oxobutan-2-yl)amino)-6-oxohexanoic acid (9)

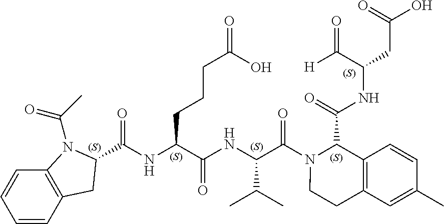

The title compound was synthesized according to the general procedure (using 150 mg resin) yielding a fluffy white solid (17 mg): RP-HPLC: 97%. ¹H NMR (300 MHz, DMSO-d₆) δ 9.31 (d, J=3.2 Hz, 1H), 8.45 (s, 1H), 8.03 (d, J=8.0 Hz, 1H), 7.82 (s, 1H), 7.22-7.09 (m, J=7.3 Hz, 2H), 7.08-6.89 (m, J=14.1, 5.9 Hz, 5H), 5.75 (s, 1H), 5.11-4.96 (m, 1H), 4.72-4.57 (m, 1H), 4.43-4.31 (m, 1H), 4.04-3.93 (m, 1H), 3.58-3.50 (m, J=10.4 Hz, 2H), 3.00-2.71 (m, 7H), 2.27-2.22 (m, 5H), 1.98 (s, 3H), 1.54-1.37 (m, 4H), 0.93-0.82 (m, 6H). HRMS (ESI-MS): m/z [M+H⁺] calculated for C₃₇H₄₅N₅O₁₀⁺: 719.3166, found 719.3256.

### Example 10. Synthesis of (S)-5-((S)-1-acetylindoline-2-carboxamido)-6-(((S)-1-((S)-1-(((S)-1-carboxy-3-oxopropan-2-yl)carbamoyl)-6-methyl-3,4-dihydroisoquinolin-2(1H)-yl)-3,3-dimethyl-1-oxobutan-2-yl)amino)-6-oxohexanoic acid (10)

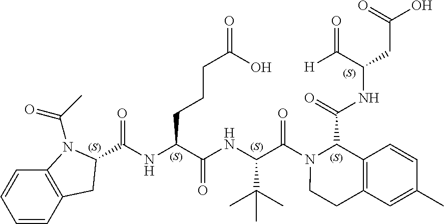

The title compound was synthesized according to the general procedure (using 150 mg resin) yielding a fluffy white solid (23 mg): RP-HPLC: 98%. ¹H NMR (300 MHz, DMSO-d₆) δ 9.30 (d, 1H), 8.02 (d, J=8.0 Hz, 1H), 7.79 (d, J=5.9 Hz, 1H), 7.29 (s, 1H), 7.17-6.89 (m, J=45.4, 8.2 Hz, 7H), 6.09-5.44 (m, J=63.0 Hz, 1H), 5.43-4.92 (m, 1H), 4.90-4.64 (m, 1H), 4.53-3.77 (m, J=78.5 Hz, 2H), 3.42-3.40 (m, 2H), 3.06-2.83 (m, 6H), 2.26 (s, 5H), 2.03-1.97 (m, 2H), 1.78-1.34 (m, 9H), 1.23 (s, 1H), 0.97 (s, 1H). HRMS (ESI-MS): m/z [M+H⁺] calculated for C₃₈H₄₇N₅O₁₀⁺: 733.3323 found 733.3413.

### Example 11. Synthesis of (S)-6-(((S)-1-((S)-1-(((S)-1-carboxy-3-oxopropan-2-yl)carbamoyl)-6-methyl-3,4-dihydroisoquinolin-2(1H)-yl)-3,3-dimethyl-1-oxobutan-2-yl)amino)-5-((S)-indoline-2-carboxamido)-6-oxohexanoic acid hydrotrifluoroacetate (11)

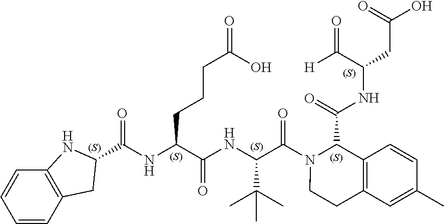

The title compound was synthesized according to the general procedure (using 150 mg resin) yielding a fluffy white solid (27 mg): RP-HPLC: 97%. ¹H NMR (300 MHz, DMSO-d₆) δ 9.31 (d, J=5.9 Hz, 1H), 8.59-8.24 (m, J 52.5 Hz, 1H), 8.02-7.86 (m, 1H), 7.68-7.38 (m, J=61.9, 8.1 Hz, 1H), 7.30-7.16 (m, J=20.6, 11.5 Hz, 1H), 7.14-6.88 (m, 5H), 6.63-6.52 (m, J=6.9 Hz, 1H), 5.95 (s, 1H), 4.92-4.76 (m, J=8.4 Hz, 1H), 4.50-4.35 (m, 1H), 4.29-4.14 (m, 1H), 4.04-3.89 (m, 1H), 3.44-3.37 (m, 2H), 3.07-2.64 (m, J=29.2 Hz, 6H), 2.28-2.16 (m, 5H), 1.80-1.45 (m, 4H), 0.98 (s, 9H). HRMS (ESI-MS): m/z [M+H⁺] calculated for C₃₆H₄₅N₅O₉⁺: 691.3217, found 691.3308.

### Example 12. Synthesis of (S)-6-(((S)-1-((S)-1-(((S)-1-carboxy-3-oxopropan-2-yl)carbamoyl)-6-methyl-3,4-dihydroisoquinolin-2(1H)-yl)-3-methyl-1-oxobutan-2-yl)amino)-5-((R)-5-fluoro-3,3-dimethylindoline-2-carboxamido)-6-oxohexanoic acid hydrotrifluoroacetate (12)

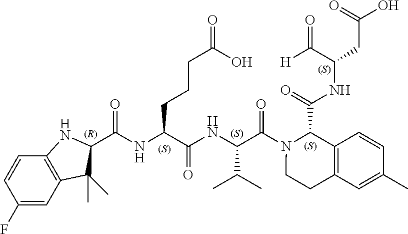

The title compound was synthesized according to the general procedure (using 150 mg resin) yielding a fluffy white solid (30 mg): RP-HPLC: 98%. ¹H NMR (300 MHz, DMSO-d₆) δ 9.19-8.89 (m, 1H), 7.67-7.53 (m, J=8.2 Hz, 1H), 7.47-7.36 (m, 1H), 7.29-7.23 (m, 1H), 7.19-6.99 (m, J=6.9 Hz, 5H), 6.64-6.44 (m, 1H), 5.63 (s, 1H), 4.60 (s, 1H), 4.45-4.35 (m, 1H), 4.30-4.17 (m, 1H), 4.00-3.91 (m, 1H), 3.45-3.40 (m, 2H), 3.05-2.77 (m, 6H), 2.20 (s, 5H), 1.78-1.52 (m, 4H), 0.81 (s, 12H). MS (ESI-M S): m/z [M−H⁻] calculated for C₃₇H₄₆FN₅O₉⁻: 722.3207, found 722.3.

### Example 13. Synthesis of (S)-6-(((S)-1-((S)-1-(((S)-1-carboxy-3-oxopropan-2-yl)carbamoyl)-6-methyl-3,4-dihydroisoquinolin-2(1H)-yl)-3-methyl-1-oxobutan-2-yl)amino)-5-((S)-5-fluoro-3,3-dimethylindoline-2-carboxamido)-6-oxohexanoic acid hydrotrifluoroacetate (13)

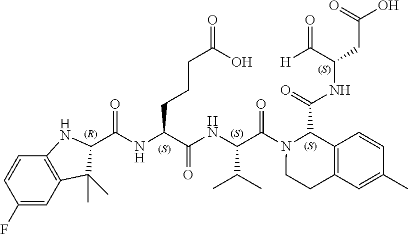

The title compound was synthesized according tote general procedure (using 150 mg resin) yielding a fluffy white solid (28 mg): RP-HPLC: 99%. ¹H NMR (300 MHz, DMSO-d₆) δ 9.29-9.15 (m, 1H), 9.10-8.71 (m, 1H), 8.72-8.39 (m, 1H), 8.37-8.06 (m, 1H), 7.80-7.16 (m, 2H), 7.13-6.40 (m, J=99.0, 35.1 Hz, 3H), 6.00-5.18 (m, 1H), 4.74-4.33 (m, J=62.7 Hz, 1H), 4.11-3.79 (m, J=47.5 Hz, 1H), 3.78-3.55 (m, 1H), 3.49-3.38 (m, 2H), 3.06-2.68 (m, J=56.4 Hz, 4H), 2.28-2.18 (m, 5H), 2.02-1.57 (m, J=98.4 Hz, 4H), 1.49-1.30 (m, J=20.2, 9.3 Hz, 6H), 0.98-0.83 (m, J=16.8 Hz, 6H). MS (ESI-MS): m/z [M−H⁻] calculated for C₃₇H₄₆FN₅O₉⁻: 722.3207, found 722.3.

### Example 14. Synthesis of (S)-6-(((S)-1-((S)-1-(((S)-1-carboxy-3-oxopropan-2-yl)carbamoyl)-5-fluoro-6-methyl-3,4-dihydroisoquinolin-2(1H)-yl)-3-methyl-1-oxobutan-2-yl)amino)-5-((S)-indoline-2-carboxamido)-6-oxohexanoic acid hydrotrifluoroacetate (14)

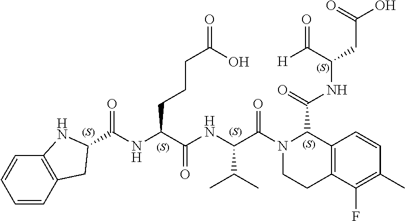

The title compound was synthesized according to the general procedure (using 150 mg resin) yielding a fluffy white solid (26 mg): RP-HPLC: 96%. ¹H NMR (300 MHz, DMSO-d₆) δ 9.32 (d, J=2.8 Hz, 1H), 9.03-8.89 (m, J=17.7 Hz, 1H), 8.25-8.12 (m, 1H), 7.89 (d, J=5.6 Hz, 1H), 7.16-7.13 (m, 1H), 7.06-6.90 (m, J=17.8, 7.8 Hz, 3H), 6.65-6.57 (m, J=7.0 Hz, 2H), 5.63 (d, J=5.0 Hz, 1H), 5.44-5.25 (m, J=23.1 Hz, 1H), 4.70-4.59 (m, 1H), 4.42-4.35 (m, 1H), 4.29-4.22 (m, J=10.5 Hz, 1H), 4.01-3.95 (m, 2H), 3.02-2.69 (m, J=40.8, 20.9 Hz, 6H), 2.36-2.24 (m, J=16.9 Hz, 1H), 2.24-2.16 (m, J=12.3 Hz, 5H), 1.69-1.46 (m, 4H), 0.92-0.83 (m, 6H). HRMS (ESI-MS): m/z [M−H⁺] calculated for C₃₅H₄₂FN₅O₉⁻: 695.2967, found 695.3049.

### Example 15. Synthesis of (S)-6-(((S)-1-((S)-1-(((S)-1-carboxy-3-oxopropan-2-yl)carbamoyl)-5-fluoro-6-methyl-3,4-dihydroisoquinolin-2(1H)-yl)-3-methyl-1-oxobutan-2-yl)amino)-5-((S)-5-fluoro-3,3-dimethylindoline-2-carboxamido)-6-oxohexanoic acid hydrotrifluoroacetate (15)

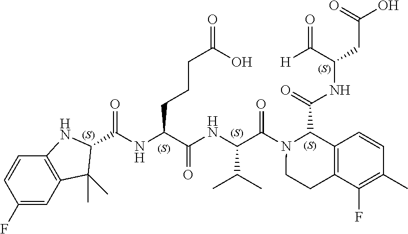

The title compound was synthesized according to the general procedure (using 450 mg resin) yielding a fluffy white solid (27 mg): RP-HPLC: 99%. ¹H NMR (400 MHz, DMSO-d₆) δ 9.32 (d, J=3.2 Hz, 1H), 9.03-8.89 (m, 1H), 8.28 (d, J=7.9 Hz, 1H), 7.77 (d, J=8.1 Hz, 1H), 7.16-7.09 (m, J=8.1 Hz, 2H), 6.91-6.75 (m, 3H), 6.62-6.57 (m, J=8.4, 4.4 Hz, 1H), 5.64 (s, 3H), 4.68-4.62 (m, 1H), 4.48-4.43 (m, 1H), 4.32-4.18 (m, J=5.8 Hz, 1H), 3.95-3.92 (m, J=6.9 Hz, 1H), 3.87 (s, 2H), 3.03-2.68 (m, 5H), 2.21-2.16 (m, 5H), 1.65-1.44 (m, 4H), 1.40 (s, 6H), 0.96 (s, 3H), 0.90 (s, 3H). MS (ESI-MS): m/z [M−H⁻] calculated for C₃₇H₄₅F₂N₅O₉⁻: 740.3113, found 740.3.

### Comparative Examples 1 and 2 (Compounds 30a and 30b)

### Comparative Example 1. Synthesis of (R)-3-((S)-2-acetamido-3-methylbutanamido)-4-(((S)-1-((S)-1-(((S)-1-carboxy-3-oxopropan-2-yl)carbamoyl)-6-methyl-3,4-dihydroisoquinolin-2(1H)-yl)-3-methyl-1-oxobutan-2-yl)amino)-4-oxobutanoic acid (30a)

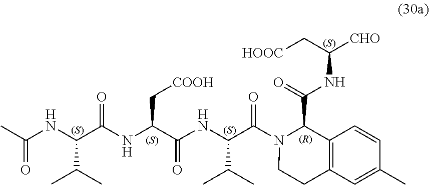

Using a procedure similar to that described in Example 1, but substituting the requisite staring materials, the title compound (30a) was prepared as a fluffy white solid (39 mg): RP-HPLC: >99%. ¹H NMR (400 MHz, DMSO-d₆) δ 9.44-8.57 (m, 1H), 8.46-8.07 (m, 1H), 8.01-7.83 (m, 1H), 7.79-7.62 (m, 1H), 7.44-7.21 (m, 1H), 7.07-6.91 (m, 2H), 6.00-5.16 (m, 2H), 4.68-4.44 (m, 2H), 4.29-4.09 (m, 1H), 4.10-3.93 (m, 2H), 3.69-3.61 (m, 214), 3.05-2.51 (m, 5H), 2.49-2.28 (m, 1H), 2.25 (s, 3H), 2.05-1.89 (m, 1H), 1.85 (s, 3H), 0.91-0.71 (m, 12H). FIRMS (ESI-MS): m/z [M+H⁺] calculated for C₃₁H₄₄N₅O₁₀⁺: 646·3083, found 646.3085.

### Comparative Example 2. Synthesis of (S)-3-((S)-2-acetamido-3-methylbutanamido)-4-(((S)-1-((S)-1-(((S)-1-carboxy-3-oxopropan-2-yl)carbamoyl)-6-methyl-3,4-dihydroisoquinolin-2(1H)-yl)-3-methyl-1-oxobutan-2-yl)amino)-4-oxobutanoic acid (30b)

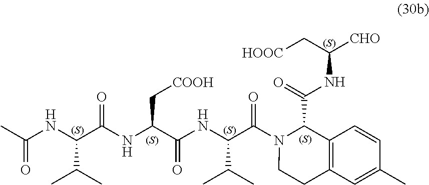

Using a procedure similar to that described in Example 1, but substituting the requisite staring materials, the title compound (30b) was prepared as a fluffy white solid (43 mg): RP-HPLC: 99%. ¹H NMR (400 MHz, DMSO-d₆) δ 9.58-8.91 (m, 1H), 8.61-8.40 (m, 1H), 8.20-8.06 (m, 1H), 7.99-7.84 (m, 1H), 7.70-7.42 (m, 1H), 7.34-7.14 (m, 2H), 6.04-5.45 (m, 2H), 4.94-4.84 (m, 1H), 4.84-4.72 (m, 1H), 4.40-4.30 (m, 2H), 4.26-4.17 (an, 2H), 3.97-3.84 (m, 1H), 3.26-2.75 (m, 5H), 2.71-2.51 (m, 1H), 2.49 (d, J=2.3 Hz, 3H), 2.32-2.20 (m, 1H), 2.20-2.11 (m, 1H), 2.09 (s, 3H), 1.17-0.99 (in, 12H). FIRMS (ESI-MS): m/z [M+H⁺] calculated for C₃₁H₄₄N₅O₁₀⁺: 646.3083, found 646.3096.

### Example 16. Identification of Selective Caspase-2 Inhibitors

### Fluorescent Enzyme Assay Protocols

### Biochemical Reagents

Human recombinant Casp1, 6, 7, and 9 were purchased from BioVision (Milpitas/CA, USA). AFC fluorogenic substrates and control peptides (AcYVAD-CHO, AcVDVAD-CHO, AcDEVD-CHO, AcVEID-CHO, and AcLEHD-CHO) were purchased from Bachem (Torrance/CA, USA). The pET23b vector encoding human caspase-3 was a gift of Dr. Michelle Arkin at University of California, San Francisco. Expression and purification of Casp3 was guided by previously described protocols (Denault J-B, Salvesen G S., Current Protocols in Protein Science, 2002, 30(1), 21.13. 1-21.13. 15). Briefly, the plasmid was transformed into E. coli Rosetta 2 pLyss DE3 cells that were cultured at 1 L scale in shake flasks. Five hours post induction with IPTG, cells were harvested through centrifugation (4000 g for 10 minutes at 4° C.), lysed by sonication, and centrifuged again for 30 minutes at 20000 g. Supernatant was resuspended in buffer (100 mM Tris pH 8.0, 500 mM NaCl). Purification was achieved by nickel-affinity chromatography followed by ion-exchange chromatography. Protein was diluted to 0.5 mg/mL and stored at −80° C. in 100 mM Tris, pH 8.0 supplemented with 10% glycerol. The typical yield for the expression, isolation, and purification was approximately 3 mg/L. The DNA encoding human caspase-2 (hCASP2, amino acids 170-452, free of the N-terminal caspase activation and recruitment domain) open reading frame (ORF) was amplified via polymerase chain reaction (PCR) from full-length hCASP2 cDNA (Origene, Rockville, MD; Cat #SC321316) using following primers: forward, 5′-ggtggtcatatgggtcctgtctgccttcaggtg-3′; reverse, 5′-ggtggtctcgagtgtgggagggtgtcctg-3′. The PCR fragment was then cloned into the pET23b vector (Millipore Sigma, Burlington, MA), and DNA sequence was verified by classic Sanger sequencing analyses. Site-directed mutageneses were carried out using a QuikChange II XL site-directed mutagenesis kit (Agilent Technologies, Santa Clara, CA; Cat #200522) according to the manufacturer's instruction. Expression of recombinant hCASP2 was induced using 150 μM isopropyl-β-D-thiogalactopyranoside (IPTG) (Promega, Madison, WI) in the BL21(DE3) E. coli strain (Millipore Sigma) at 16° C. for 14 hours while shaking at 250 rpm. Cells were then harvested via centrifugation at 6,000 g, 4° C. for 15 minutes, followed by lysis via sonication. Proteins were initially purified using HisPur Ni-NTA resin (Thermo Fisher Scientific, Waltham, MA) followed by metal chelating chromatography (5 mL HiTrap Chelating HP columns (GE Healthcare Life Sciences, Piscataway, NJ)). Further purification was carried out using anion exchange chromatography (5 mL HiTrap Q HP columns (GE Healthcare Life Sciences)) for hCASP2. Proteins were stored in 1× phosphate-buffered saline (pH 7.4) at 10 mg/mL (concentration determined using a BCA assay (Thermo Fisher Scientific)), −80° C. until further use.

### 384-Well Protocol

K_{M} values were determined experimentally to be the following: Casp1: 5.9 μM; Casp2: 37.1 μM; Casp3: 7.6 μM; Casp6: 14.5 μM; Casp7: 13.8 μM; Casp9: 149.1 μM. Enzymes were diluted in buffer: 100 mM MES (pH 6.5) for Casp2 and or 100 mM HEPES (pH 7.0) for all other caspases, plus 150 mM NaCl, 0.1% CHAPS, 1.5% sucrose, 10 mM DTT. Enzyme concentrations were 0.05 U/well for Casp1, 6, and 7; 0.5 U/well for Casp9, 20 nM/well for Casp2, and 2 nM/well for Casp3. Enzyme in buffer (19 μL) was added per well in a black 384-well Corning 4514 assay plate. Test compounds were serially diluted in dimethyl sulfoxide (DMSO) and plated in duplicate into a Corning 3656 transfer plate. Test compound was added to assay plates in 0.5 μL aliquots per well and mixed 10 times using a BiomekFX (Beckman Coulter). Compound and enzyme mixture was incubated at 37° C. for 5 minutes for aldehyde warheads and 45 minutes for cyano warheads. The BiomekFX was then used to add and mix 0.5 μL of the AFC substrate in DMSO from a Corning 3656 transfer plate (final assay concentrations: 5 μM AcYVAD-AFC for Casp1, 10 μM Z-VDVAD-AFC for Casp2, 5 μM AcDEVD-AFC for Casp3, 5 μM Z-VEID-AFC for Casp6, 5 μM AcDEVD-AFC for Casp7, and 34 μM AcLEHD-AFC for Casp9) to the assay plate for a total assay volume of 20 μL. Fluorescence from free AFC was read at 37° C. every 5 minutes over an hour using a Clariostar plate reader (BMG Labtech) (λex=400 nm, λem=505 nm). The 40-minute time point was reported.

### 96-Well Protocol (Casp2/3)

Enzyme was diluted in buffer: 100 mM MES (pH 6.5) for Casp2 or 100 mM HEPES (pH 7.0) for Casp3, plus 150 mM NaCl, 0.1% CHAPS, 1.5% sucrose, and 10 mM DTT. Enzyme concentrations were 5 nM/well for Casp2 and 2 nM/well for Casp3. Enzyme in buffer (96.5 μL) was added per well in a black Corning 3356 96-well assay plate. Test compounds were serially diluted in dimethyl sulfoxide (DMSO) and plated in triplicate in a Corning 3357 transfer plate. Test compound was added to assay plates in 1 μL aliquots per well and mixed 10 times using a BiomekFX (Beckman Coulter). Compound and enzyme mixture was incubated at 37° C. for 5 minutes for aldehyde warheads and 45 minutes for cyano warheads. The BiomekFX was then used to add and mix 2.5 μL of the AFC substrate in DMSO from a transfer plate (final assay concentrations: 25 μM Z-VDVAD-AFC for Casp2, 10 μM AcDEVD-AFC for Casp3) to the assay plate for a total assay volume of 100 μL in the assay plate. Fluorescence from free AFC was read at 37° C. every 5 minutes over an hour using a Clariostar plate reader (BMG Labtech) (λex=400 nm, λem=505 nm). The 40-minute time point was reported, consistent with reported literature.

Data is shown in the following Table 1.

TABLE 1
Binding data (pKᵢ values) of pentapeptides 1-15 and 30a/b at Casp2
and Casp3. Selectivity expressed as Kᵢ (Casp3) / Kᵢ (Casp2).

| | pKᵢ ± SEM | | | | Kᵢ (Casp3) / Kᵢ |
|---|---|---|---|---|---|
| Cmpd. | Casp 2 | N | Casp 3 | N | (Casp2) |
| 1 | 8.12 ± 0.10 | 3 | 6.03 ± 0.06 | 3 | 123 |
| 30a | 4.98 ± 0.20 | 5 | <4 | 4 | >9 |
| 30b | 7.89 ± 0.02 | 5 | 6.04 ± 0.06 | 5 | 70 |
| 2 | 7.40 ± 0.11 | 3 | <4.5 | 3 | 794 |
| 3 | <4 | 3 | <4 | 3 | — |
| 4 | <4 | 3 | <4 | 3 | — |
| 5 | 7.77 ± 0.11 | 5 | 5.26 ± 0.24 | 5 | 324 |
| 6 | 5.64 ± 0.07 | 3 | 4.48 ± 0.05 | 3 | 14 |
| 7 | 7.53 ± 0.15 | 3 | <4 | 3 | 3388 |
| 8 | 5.22 ± 0.06 | 3 | <4 | 3 | 17 |
| 9 | 6.42 ± 0.03 | 3 | <4 | 3 | 263 |
| 10 | 5.67 ± 0.02 | 3 | <4 | 3 | 47 |
| 11 | 5.38 ± 0.25 | 3 | <4 | 3 | 24 |
| 12 | 5.38 ± 0.01 | 2 | <4 | 3 | 24 |
| 13 | 6.19 ± 0.01 | 2 | <4 | 3 | 155 |
| 14 | 6.72 ± 0.05 | 3 | <4 | 3 | 525 |
| 15 | 6.76 ± 0.05 | 2 | <4 | 3 | 575 |

Data shown are mean values ± SEM of N independent experiments, each performed in duplicate or triplicate.
Data were analyzed by nonlinear regression and were best fitted to sigmoidal concentration-response curves.

Compound 1 showed high-affinity at Casp2 (pKᵢ=8.12; cf. Table 1), a promising selectivity profile in an in vitro caspase panel assay (123-fold selective versus Casp3 and >2000-fold selective versus Casp1, 6, 7, 9; cf. Table 2) and, evinced only weak activity at thrombin and cathepsin B proteolytic enzymes (See Table 2).

### Example 17. Compound 1 Blocks Tau Truncation and Restores Excitatory Neurotransmission in Neurons Modeling FTDP-17 Tauopathy

### Results

In an in vitro assay, Compound 1 was shown to effectively inhibit Casp2-mediated cleavage of tau and to block the production of Δtau314, the N-terminal truncation product formed by hydrolysis at aspartate-314. Using cultured rat and mouse hippocampal neurons expressing P301S tau, Compound 1 was found to prevent the disproportionate accumulation of P301S tau in dendritic spines. Finally, it was found that the expression of P301S tau impairs postsynaptic function of excitatory synapses. This was determined by recording miniature excitatory synaptic currents (mEPSCs). Compound 1 was found to completely normalize mEPSCs and to rescue synaptic function.

### Casp2-Selective Inhibitors Block Enzyme-Catalyzed Tau Cleavage at D314.

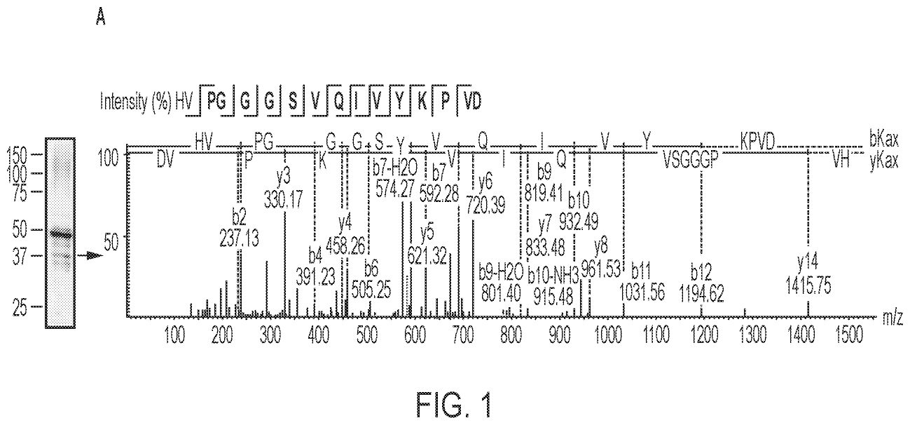

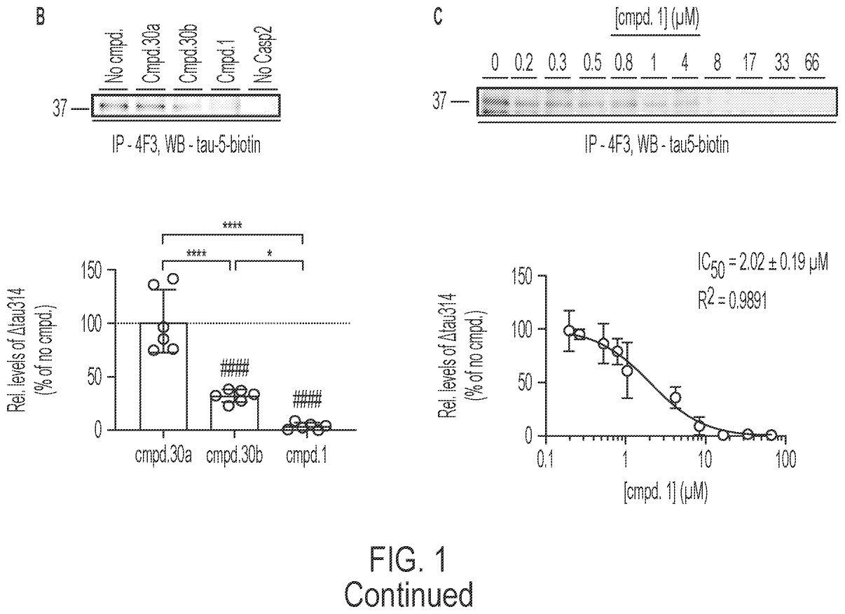

To understand the effects of Casp2-selective inhibitors on enzyme-catalyzed site-specific cleavage of tau, a naturally occurring substrate in the brain, in vitro Casp2 cleavage assays were performed using purified recombinant proteins. The presence of Δtau314, the soluble truncated tau fragment ending C-terminally at D314 (Zhao X, et al., Nat Med., 2016, 22, 1268-76), was verified by mass spectrometry (MS) (FIG. 1A). Both Casp2-selective compounds, 1 and 30b, inhibited enzyme-catalyzed Δtau314 production at 10 μM but with different efficacies (96% for 1 versus 68% for 30b). In contrast, 30a, the epimer of 30b, did not inhibit Δtau314 production at 10 μM (FIG. 1B). 1 inhibited tau-cleavage at D314 with an IC₅₀ of 2.02 μM (FIG. 1C).

### Compound 1 Prevents the Disproportionate Accumulation of P301S Tau in Dendritic Spines of Cultured Rat Hippocampal Neurons.

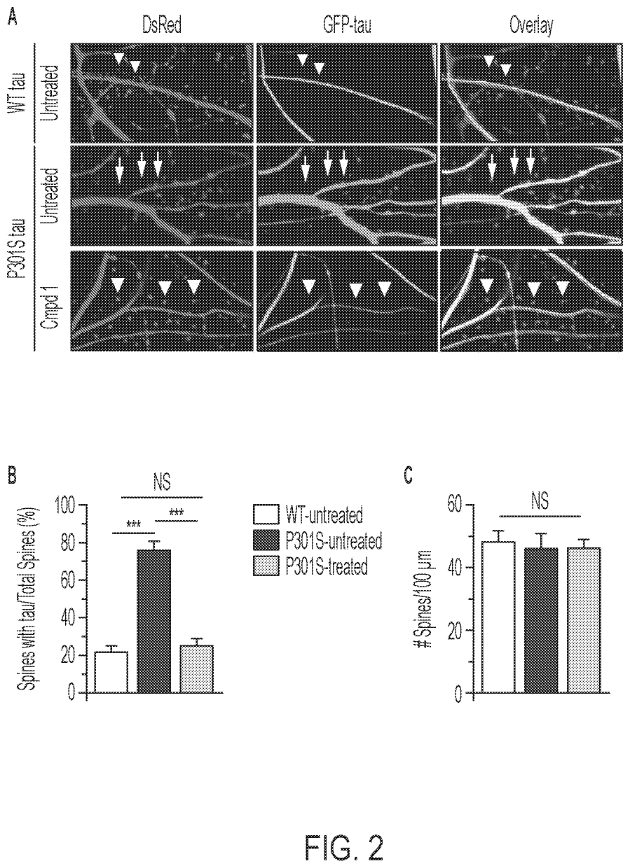

The disproportionate accumulation in dendritic spines of mutant tau linked to FTDP-17 depends upon tau-truncation by Casp2 (Zhao X, et al., Nat Med., 2016, 22, 1268-76). As shown in FIG. 1, of the compounds tested the one that blocks tau-truncation most effectively is Compound 1. The P301S tau mutation linked to FTDP-17 (Bugiani O, et al., J Neuropathol Exp Neurol., 1999, 58, 667-677; and Lossos A, et al., J Neurol., 2003, 250, 733-740) was examined to determine whether Compound 1 could prevent the accumulation of tau in dendritic spines. Dissociated hippocampal neurons from postnatal day 0-1 rat pups were cultured. After 5-7 days in vitro (DIV), the neurons were co-transfected with plasmids encoding DsRed (to label neuronal morphology) and GFP-tagged wild-type or P301S mutant tau proteins (FIG. 2A), as previously described (Hoover B R, et al., Neuron., 2010, 68:1067-1081). Neurons at 17-18 DIV were incubated with 10 μM Compound 1 for 3 days and live neurons were imaged at 20-21 DIV.

As shown in FIG. 2A, in neurons co-expressing DsRed and wild-type tau, relatively few dendritic spines contain GFP-tagged tau proteins (top row). In contrast, in neurons co-expressing DsRed and GFP-tagged P301S tau, most of the dendritic spines contain GFP-tagged tau (middle row). When 1 was applied to neurons expressing GFP-tagged P301S tau, a minority of dendritic spines contain GFP-tagged tau (bottom row). The percentage of dendritic spines containing GFP-tagged tau proteins in neurons significantly increased from 21.9% in neurons expressing wild-type tau to 75.5% in neurons expressing P301S tau; Compound 1 reduced the percentage to 24.8% (FIG. 2B; n=8 neurons in each group; ANOVA, F=82.1; Bonferroni post-hoc test: P<0.01 comparing untreated wild-type and P301S tau, and then comparing untreated and treated P301S tau). There were no significant differences in the density of dendritic spines between the three groups (FIG. 2C; ANOVA, F=0.13, P>0.05), indicating that neither the P301S mutation nor Compound 1 caused loss of dendritic spines. Overall, these results indicate that Compound 1 blocks the excessive accumulation of tau induced by the P301S mutation in dendritic spines, supporting its efficacy in reversing the molecular pathology of dendritic spines in a cellular model of tauopathy.

### Compound 1 Rescues P301S Tau-Induced Functional Deficits in Dendritic Spines.

Previous studies in models of several different tauopathies demonstrated that the excessive accumulation of tau in dendritic spines is associated with a reduction in postsynaptic excitatory neurotransmission due to the internalization of AMPA receptors (Hoover B R, et al., Neuron., 2010, 68:1067-1081; Terayskis P J, et al., Journal of Neuroscience, 2018, 38, 9754-67; Singh B, et al., Acta Neuropathologica, 2019, 138, 551-74; Zhao X, et al., Nat Med., 2016, 22, 1268-76; Miller E C, et al., Eur J Neurosci, 2014, 39, 1214-24; Braun N J, et al., Proc Natl Acad Sci USA., 2020, 117, 29069-29079; and Terayskis P J, et al., Journal of Physiology (Oxford, United Kingdom), 2021, 599, 2483-98). Therefore, the abilities of Compounds 1, 30a, and 30b to rescue functional synaptic deficits in a cellular model of tauopathy were compared.

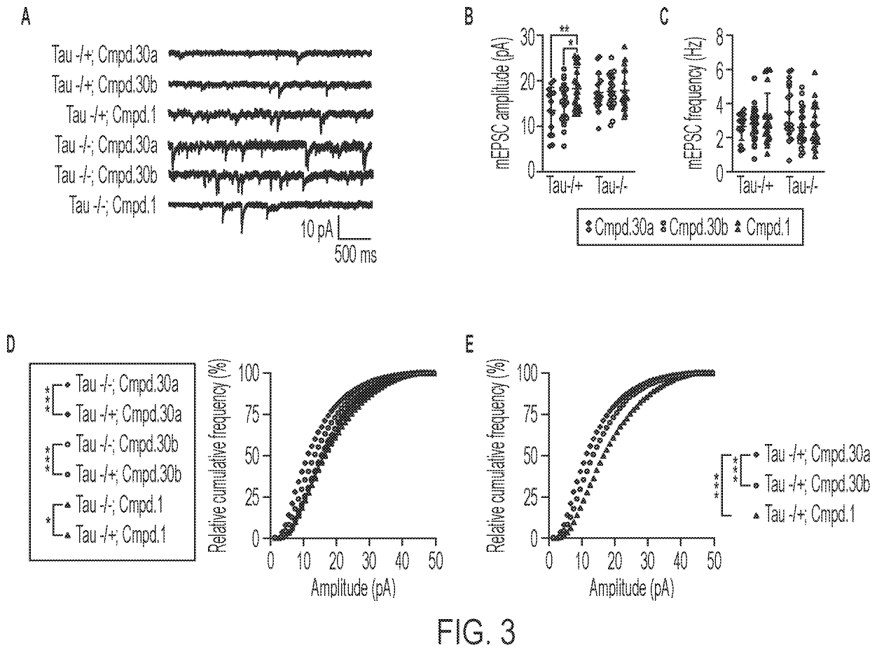

For these studies, PS19 transgenic mice expressing P301S mutant tau, one of the most used models of FTDP-17 were used (Feinstein S. Faculty Opinions recommendation of Synapse loss and microglial activation precede tangles in a P301S tauopathy mouse model. Faculty Opinions—Post-Publication Peer Review of the Biomedical Literature. 2007; and Takeuchi H, et al., PLoS One., 2011, 6, e21050). To detect functional deficits, primary hippocampal neurons were cultured from heterozygous transgenic mice overexpressing P301S tau (Feinstein S. Faculty Opinions recommendation of Synapse loss and microglial activation precede tangles in a P301S tauopathy mouse model. Faculty Opinions—Post-Publication Peer Review of the Biomedical Literature. 2007; and Takeuchi H, et al., PLoS One., 2011, 6, e21050) and littermate controls, (denoted Tau−/+ and Tau−/− respectively in FIG. 3), and recorded miniature excitatory synaptic currents (mEPSCs). 1, 30a (inactive analog of 30b), or 30b (final concentrations=10 μM) were directly applied to the culture media (FIG. 3A) and incubated for 3 days. In the two groups treated with Compound 30a, the average amplitude of mEPSC responses in Tau−/+ neurons is significantly smaller than that in Tau−/− neurons (FIG. 3B, ANOVA, F=3.3; Bonferroni post hoc test: P<0.05), indicating that the expression of P301S tau impaired postsynaptic function of the excitatory synapses by reducing the number of functional AMPA receptors on post-synaptic membranes. In contrast, the average amplitudes of mEPSC responses in Tau−/+ and Tau−/− neurons treated with Compound 1 do not differ significantly, suggesting that Compound 1 rescues impaired mEPSC amplitudes (ANOVA, F=3.3, P>0.05). The average amplitudes of mEPSC responses of Tau−/+ neurons treated with Compound 1 are significantly larger than those treated with Compound 30a (FIG. 3B, F=3.3; Bonferroni After Test: P<0.01) or 30b (FIG. 3B, F=3.3; Bonferroni post-hoc test: P<0.05), indicating that Compound 1 rescues post-synaptic neurotransmission more effectively than Compound 30a or 30b. There were no significant differences in the average frequency of mEPSCs between the six groups (FIG. 3C, ANOVA, F=0.6, P>0.05), indicating no changes in presynaptic function.

To confirm and extend the results in FIG. 3B, the cumulative curves of mEPSC amplitudes were analyzed. The cumulative curve of mEPSC amplitudes of Compound 30a-treated neurons from Tau−/+ compared with Tau−/− mice is shifted to the left (FIG. 3D, Mann-Whitney U test: P<0.001), indicating that the P301S mutation decreases the amplitudes of mEPSCs. The cumulative curve of mEPSC amplitudes of Compound 30b-treated neurons from Tau−/+ compared with Tau−/− mice is shifted to the left but less markedly (FIG. 3D, Mann-Whitney U test: P<0.001), indicating that Compound 30b partially blocks the synaptic impairment caused by the P301S mutation. In contrast, the cumulative curve of mEPSC amplitudes of Compound 1-treated neurons from Tau−/+ compared with Tau−/− mice is not significantly shifted (FIG. 3D, Mann-Whitney U test: P>0.05), which indicates that Compound 1 completely blocks the synaptic impairment caused by the P301S mutation. Comparison of the cumulative curves of Tau−/+ neurons treated with each of the three compounds indicates that Compound 1 shifted the cumulative curve of mEPSC amplitudes more to the right than Compound 30b, further supporting that Compound 1 is the more effective compound (FIG. 3E, Mann-Whitney U test: P<0.001).

### Discussion

Compound 1, a new, potent, and selective Casp2 inhibitor, is significantly more effective than Compound 30b in restoring excitatory postsynaptic neurotransmission in a cellular model of tauopathy.

Compound 1 rescues tau-induced synaptic dysfunction by blocking the excessive accumulation of tau in dendritic spines (FIGS. 3 and 4). This mechanism has been shown to mediate synaptic dysfunction repeatedly in models of tauopathy ranging from Alzheimer's disease, Lewy body dementia, and FTDP-17 to chronic traumatic encephalopathy (Hoover B R, et al., Neuron., 2010, 68:1067-1081; Terayskis P J, et al., Journal of Neuroscience, 2018, 38, 9754-67; Singh B, et al., Acta Neuropathologica, 2019, 138, 551-74; Zhao X, et al., Nat Med., 2016, 22, 1268-76; Miller E C, et al., Eur J Neurosci, 2014, 39, 1214-24; Braun N J, et al., Proc Natl Acad Sci USA., 2020, 117, 29069-29079; and Terayskis P J, et al., Journal of Physiology (Oxford, United Kingdom), 2021, 599, 2483-98). The dysfunctional process begins when tau redistributes from axons to dendrites in response to various pathological triggers (Terayskis P J, et al., Neuroscientist, 2020, 26, 503-20; and Tracy T E, et al., Curr Opin Neurobiol., 2018, 51, 34-8). Our current working model stipulates that the phosphorylation of serine-396 or serine-404 in the C-terminal tail of tau by GSK3β and/or CDK5 concomitantly with the cleavage of tau by Casp2 at aspartate-314 in the third microtubule domain causes tau to accumulate in dendritic spines. A minority of dendritic spines normally contains tau (Kimura T, et al., Philosophical Transactions of the Royal Society B: Biological Sciences, 2014, 369, 20130144), but under pathological conditions tau is found in the vast majority of dendritic spines. This occurs presumably because non-physiological, pathological triggers activate or elevate the levels of Casp2 and relevant kinases. The presence of tau in dendritic spines stimulates calcineurin-dependent endocytosis of AMPARs, but only if one or more of five specific residues in the proline-rich domain of tau are phosphorylated.

Elevated levels of Δtau314 and Casp2 found in brain specimens of individuals with Alzheimer's disease (Zhao X, et al., Nat Med., 2016, 22, 1268-76; and Liu W, et al., Translational Psychiatry, 2020; 10), Lewy body dementia (Liu, P, et al., Acta Neuropathologica Communications, 2019, 7, 124), and Huntington's disease (Liu, P, et al., Acta Neuropathologica Communications, 2019, 7, 111) underscore the relevance of the Casp2-tau signaling pathway in tauopathies. The results reported here using Compound 1 establish the feasibility of using a small-molecule Casp2 inhibitor to restore excitatory neurotransmission and to treat patients with dementia due to tauopathies.

### Materials and Methods

### Animal Care and Usage.

Rat and mouse pups in postnatal day 0 or 1 were harvested to make primary hippocampal cultures. Sprague-Dawley timed-pregnancy adult rats were housed in facilities of Research Animal Resources (RAR) at the University of Minnesota (UMN) after being delivered from Envigo (www.envigo.com). The rats were fed a diet of regular chow before giving birth. Rat pups were euthanized by decapitation to harvest brain tissues in postnatal day 0 or 1. The appropriate transgenic mice were also housed and bred in RAR facilities at UMN. All work was conducted in accordance with the American Association for the Accreditation of Laboratory Animal Care and Institutional Animal Care and Use Committee (IACUC) at the University of Minnesota (protocol #1211A23505). We performed all procedures of euthanasia strictly according to the guidelines of the IACUC at the UMN.

### Materials

### Expression and Purification of Recombinant Caspase-2 and Tau for the In Vitro Caspase-2-Catalyzed Tau Cleavage Assay

DNA sequences encoding human caspase-2 p19 and p12 subunits were cloned as two separate open reading frames in the pCOLADuet-1 vector (Millipore Sigma, Burlington, MA). The DNA sequence encoding human microtubule-associated protein tau 0N4R isoform was cloned in the pET28a vector (Millipore Sigma). Expression of recombinant human caspase-2 and tau was induced using 150 μM isopropyl-β-D-thiogalactopyranoside (IPTG) (Promega, Madison, WI) in the BL21(DE3) E. coli strain (Millipore Sigma) at room temperature for 16 hours while shaking at 250 rpm. Cells were then harvested via centrifugation at 6,000 g, 4° C. for 15 minutes, followed by lysis via sonication. Proteins were purified using HisPur Ni-NTA resin (Thermo Fisher Scientific, Waltham, MA) followed by HiTrap Chelating HP columns (GE Healthcare Life Sciences, Piscataway, NJ). Proteins were stored in 1× phosphate-buffered saline (pH 7.4) at 10 mg/mL (determined using a BCA assay (Thermo Fisher Scientific)), −80° C. until further use.

Statistical analyses for the in vitro caspase-2-catalyzed tau cleavage assay were performed using GraphPad Prism Version 8.3.0 (GraphPad Software, La Jolla, CA). P<0.05 was considered statistically significant. Experiments were repeated six times. Individual values (open circles), means (histograms), and standard deviations (SDs, error bars) are shown. A single sample t-test was performed to compare the effect of each compound to that of no compound (####, p<0.0001). One-way ANOVA was performed to compare effects of tested compounds (F_{(2, 15)}=48.50, P<0.0001), followed by Tukey's post hoc test (*, p<0.05; ****, p<0.0001).

Determination of the IC₅₀ of compound 1. Upper panel, a representative IP (4F3)/WB (tau-5-biotin) showing the produced Δtau314 in the presence of various concentrations of compound Lower panel, levels of Δtau314 (normalized to those produced with no compound) and compound concentrations were fit into a dose-response curve with the IC₅₀ of compound 1 determined as 2.02±0.19 μM. Experiments were performed in duplicates. Means (open circles) and SDs (error bars) are shown.

### Antibody Immobilization on Protein G Magnetic Beads

The 4F3 antibody was covalently linked to Protein G magnetic beads as previously described by Liu P, et al., Acta Neuropathologica Communications, 2019, 7(1), 1-13.

### Preparation of Samples for Mass Spectrometry

Purified recombinant tau (1 mg) were incubated with purified recombinant caspase-2 at a molar ratio of 1:1 under the conditions described in the “In vitro caspase-2-catalyzed tau cleavage assay” section. The resulting material was diluted in an IP buffer (final volume: 1 mL) and incubated overnight at 4° C. with 4F3-bound Protein G-coupled magnetic beads. Following resin wash and protein elution, the immunocaptured proteins were size-fractionated, and the gel area containing proteins of interest was isolated using a protocol described by Liu W, et al., Translational Psychiatry, 2020, 10(1), 1-9.

### Plasmids and PCR Mutagenesis

For all cultured hippocampal neuron studies all human tau and DsRed constructs were expressed in the pRK5 vector and driven by the cytomegalovirus promoter (Clontech Inc.). Human tau proteins were N-terminally fused to enhance GFP (eGFP). The wild-type, native human tau construct encoded human four-repeat tau lacking the transcriptional-variant N-terminal sequences (0N4R) and contained exons 1, 4, 5, 7, 9-13, 14, and intron 13 (RRID: Addgene_46904). The P301S mutant was created using site-directed mutagenesis (QuikChange SDM Kit, Agilent). PCR primers with lengths of 31 and 28 nucleotides were used for mutagenesis (sense: 5′-GCCGCCTCCCGaGACGTGTTTGATATTATCC-3′; antisense: 5′-TATCAAACACGTCtCGGGAGGCGGCAGT-3′; mutated nucleotide represented as lower case letter) (Integrated DNA Technologies). The nucleotide mutation as well as plasmid construct integrity were confirmed with Sanger Sequencing (UMN Genomics Center). Tau sequence numbering was based on the longest functional human isoform: 441-tau (2N4R tau; NCBI reference sequence: NP_005901.2).

### Primary Hippocampal Neuron Cultures

Briefly, a 22 mm diameter glass coverslip (0.09 mm thickness) was silicone-sealant-fastened to the bottom of a 35 mm culture dish with a bored hole of 20 mm in diameter and sterilized as described by Smith B R, et al., Acta Neuropathologica Communications, 2019, 7(1), 124. Coverslips were coated with poly-D-lysine. Hippocampi were dissected from neonatal Sprague-Dawley timed-pregnancy rats (Envigo) or appropriate transgenic mice and control littermates (see main text) at post-natal day 0-1. Hippocampi were enzymatically digested in Earle's Balanced Salt Solution (EBSS) supplemented with 1% glucose and cysteine-activated papain. Digestion was blocked with dilute BSA (bovine serum albumin) and chicken ovomucoid, and cells were rinsed in fresh EBSS and plated in plating medium (minimal essential medium with Earle's salts, 10% fetal bovine serum, 5% horse serum, 2 mM glutamine, 10 mM sodium pyruvate, 0.6% glucose, 100 U/ml penicillin and 100 mg/ml streptomycin) at 1.0×10⁶ cells/dish. After 18 h cell adherence was established. Cells were then grown in a neurobasal medium (a 1:5 mixture of NbActiv4 to NbActiv1; BrainBits LLC) and incubated at 37° C. in a 5% CO₂ biological incubator.

### Low Efficiency Calcium-Phosphate Transfection

At 5-7 days in vitro (DIV) cells were transfected. DNA plasmid transfection was performed using standard calcium phosphate precipitation and incubation as described by Liu P, et al., Acta Neuropathologica Communications, 2019, 7(1), 1-13 Briefly, neurons were transfected with human tau constructs and DsRed (2:1 by plasmid DNA mass) for live imaging, and with human tau alone for electrophysiology and immunocytochemistry. Precipitated DNA was applied to cells in a solution of NbActiv4 neurobasal medium containing 100 μM AP5 ((2R)-amino-5-phosphovaleric acid) to prevent calcium-induced excitotoxicity. After 3-4 hours of transfection time, cells were rinsed in a glial conditioned medium and grown in the neurobasal medium as described above until mature (21-28 DIV).

### In Vitro Caspase-2-Catalyzed Tau Cleavage Assay.

Purified recombinant Casp2 (final concentration during pre-incubation: 67 μM) was pre-incubated with various concentrations of inhibitory compounds at 4° C. for 72 h. Pre-treated enzyme was then incubated with purified recombinant tau at a molar ratio of 1:1 in 37° C. water bath, 1× reaction buffer (25 mM 4-(2-hydroxyethyl)-1-piperazineethanesulfonic acid (HEPES), 0.1% (w/v) 3-[(3-cholamidopropyl)dimethylammonio]-1-propanesulfonate (CHAPS), 10 mM dithiothreitol (DTT), pH 7.5) for 7 h. The final volume of each reaction was 100 μL. At the end of the 7-h incubation, 0.1% (v/v) protease inhibitor cocktail (Millipore Sigma) was added to stop the reactions.

### Immunoprecipitation (IP)/Western Blotting (WB).

Immediately after the reaction was stopped, the 100 μL enzyme-product mixture was diluted in 400 μL IP buffer (50 mM Tris-HCl (pH 7.4) and 150 mM NaCl, containing 0.1 mM phenylmethylsulfonyl fluoride, 0.2 mM 1,10-phenanthroline monohydrate, and protease inhibitor cocktail (Millipore Sigma)), and then incubated with 10 μg of Δtau314-specific monoclonal antibody 4F3 and 50 μL of Protein G Sepharose 4 Fast Flow resin (GE Healthcare) at 4° C. for 14-16 h. Subsequent resin wash and protein elution were performed as described previously (Liu P, et al., Scientific Reportsm 2020, 10, 3869). WB was performed according to a previously published protocol (Liu P, et al., Acta Neuropathologica Communications, 2019; 7, 111).

### Mass Spectrometry (MS).

In-gel trypsin digestion, liquid chromatography-MS/MS, mass spectral database search, and data interpretation were performed as previously described Liu W, et al., Translational Psychiatry, 2020; 10); except that Peaks Studio Xpro (Bioinformatics Solutions, Inc, Waterloo, Ontario, Canada) was used for interpretation of mass spectra.

### Electrophysiology.

Miniature excitatory postsynaptic currents (mEPSCs) were recorded from cultured dissociated mouse hippocampal neurons at 17-21 DIV with a glass pipette (resistance ˜5 MΩ) at holding potentials of −65 mV on an Axopatch 200B amplifier (output gain=0.5; filtered at 1 kHz) as we previously described (Miller E C, et al., Eur J Neurosci., 2014, 39, 214-224). Input and series resistances were assessed and found to have no significant difference before and after recording time (5-20 mins). Recording sweeps lasted 200 ms and were sampled for every 1 s (pClamp, v10, RRID: SCR_011323). Neurons were bathed in bubble-oxygenated artificial cerebrospinal fluid (ACSF) at 23° C. with 100 μM APV (NMDA receptor antagonist), 1 μM TTX (sodium channel blocker), and 100 μM picrotoxin (GABAa receptor antagonist). Passive oxygen perfusion was established with medical-grade 95% O₂-5% CO₂. ACSF contained (in mM) 119 NaCl, 2.5 KCl, 5.0 CaCl₂), 2.5 MgCl₂, 26.2 NaHCO₃, 1 NaH₂PO₄, and 11 D-glucose. The internal solution of the glass pipettes contained (in mM) 100 cesium gluconate, 0.2 EGTA, 0.5 MgCl₂, 2 ATP, 0.3 GTP, and 40 HEPES. The pH of the internal solution was normalized to 7.2 with cesium hydroxide and diluted to a trace osmotic deficit in comparison to ACSF (˜300 mOsm). All analysis of recordings was performed using an automated detection software suit (Clampfit, 11.0.3, Molecular Devices, San Jose, CA, USA). Minimum analysis parameters were set at greater than 1 min stable recording time and events with amplitudes greater than 3 pA and smaller than 40 pA were included. A mEPSC event was identified by using a template which included a distinct fast-rising depolarization and slow-decaying repolarization. Combined individual events were used to form relative cumulative frequency curves; whereas the means of all events from individual recordings were treated as single samples for further statistical analysis. Example traces were exported from Clampfit and live-traced, simplified, and united in vector editing software (Adobe Illustrator CS5 and Affinity Designer).

### Example 18. The Following Illustrate Representative Pharmaceutical Dosage Forms, Containing a Compound of Formula (I) (‘Compound X’), for Therapeutic or Prophylactic Use in Humans

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | mg/tablet |
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X= (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | |
| (pH adjustment to 7.0-7.5) | q.s. |
| Water for injection | q.s. ad 1 mL |
| (v) Injection 2 (10 mg/ml) | mg/ml |
| Compound X= (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | |
| (pH adjustment to 7.0-7.5) | q.s. |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound of Formula (I): 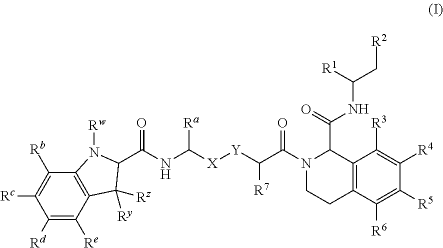
or a salt thereof, wherein:
R¹ is —B(OH)₂, cyano, (C₁-C₆)alkyl, (C₂-C₆)alkenyl, or (C₂-C₆)alkynyl, wherein each (C₁-C₆)alkyl, (C₂-C₆)alkenyl, and (C₂-C₆)alkynyl is optionally substituted with one or more groups independently selected from the group consisting of halo, cyano, oxo, NR^{r}R^{s}, and aryl, wherein each aryl is optionally substituted with one or more groups independently selected from the group consisting of halo, hydroxy, (C₁-C₆)alkyl, cyano, (C₁-C₆)alkoxy, (C₁-C₆)alkoxycarbonyl, carboxy, and (C₁-C₆)alkanoyloxy, and wherein one or more sp3 carbon atoms of each (C₁-C₆)alkyl, (C₂-C₆)alkenyl, and (C₂-C₆)alkynyl is optionally replaced with O, S, S(O), S(O)₂, or NH; or R¹ is —C(═O)R^{p};
R² is —C(═O)OR^{f} or —C(═O)NR^{g}R^{h};
R³ is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R⁴ is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R⁵ is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R⁶ is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R⁷ is H or (C₁-C₆)alkyl that is optionally substituted with (C₁-C₆)alkoxy or (aryl)(C₁-C₆)alkoxy;
R^{a} is H or (C₁-C₆)alkyl that is substituted with —C(═O)OR^{k} or —C(═O)NR^{m}Rⁿ;
R^{b} is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy
R^{c} is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy
R^{d} is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R^{e} is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy
R^{f} is selected from the group consisting of H, (C₁-C₆)alkyl, (C₃-C₆)cycloalkyl, (C₃-C₆)cycloalkyl(C₁-C₆)alkyl, and aryl(C₁-C₆);
each R^{g} and R^{h} is independently selected from the group consisting of H, (C₁-C₆)alkyl, (C₃-C₆)cycloalkyl, (C₃-C₆)cycloalkyl(C₁-C₆)alkyl, and aryl(C₁-C₆); or R^{g} and R^{h} taken together with the nitrogen to which they are attached form an aziridino, azetidino, morpholino, piperazino, pyrrolidino or piperidino, which aziridino, azetidino, morpholino, piperazino, pyrrolidino or piperidino is optionally substituted with one or more groups independently selected from the group consisting (C₁-C₆)alkyl;
R^{k} is selected from the group consisting of H, (C₁-C₆)alkyl, (C₃-C₆)cycloalkyl, (C₃-C₆)cycloalkyl(C₁-C₆)alkyl, and aryl(C₁-C₆);
each R^{m} and Rⁿ is independently selected from the group consisting of H, (C₁-C₆)alkyl, (C₃-C₆)cycloalkyl, (C₃-C₆)cycloalkyl(C₁-C₆)alkyl, and aryl(C₁-C₆); or R^{m} and Rⁿ taken together with the nitrogen to which they are attached form an aziridino, azetidino, morpholino, piperazino, pyrrolidino or piperidino, which aziridino, azetidino, morpholino, piperazino, pyrrolidino or piperidino is optionally substituted with one or more groups independently selected from the group consisting (C₁-C₆)alkyl;
R^{p} is a detectable group;
each R^{r} and R^{s} is independently selected from the group consisting of H, (C₁-C₆)alkyl, (C₃-C₆)cycloalkyl, (C₃-C₆)cycloalkyl(C₁-C₆)alkyl, and aryl(C₁-C₆); or R^{r} and R^{s} taken together with the nitrogen to which they are attached form an aziridino, azetidino, morpholino, piperazino, pyrrolidino or piperidino, which aziridino, azetidino, morpholino, piperazino, pyrrolidino or piperidino is optionally substituted with one or more groups independently selected from the group consisting (C₁-C₆)alkyl;
X is C═O, C═S, or —CH₂—;
Y is —CH₂— or —NH—;
R^{w} is H or (C₁-C₆)alkanoyl;
R^{y} is H or (C₁-C₆)alkyl; and
R^{z} is H or (C₁-C₆)alkyl.

2. The compound or salt of claim 1, which is a compound of formula (Ia): 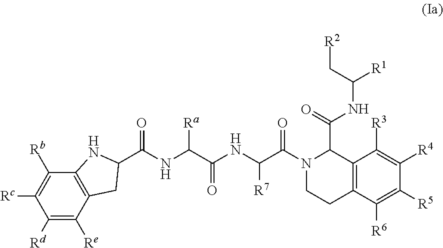
or a salt thereof, wherein:
R¹ is —B(OH)₂, cyano, (C₁-C₆)alkyl, (C₂-C₆)alkenyl, or (C₂-C₆)alkynyl, wherein each (C₁-C₆)alkyl, (C₂-C₆)alkenyl, and (C₂-C₆)alkynyl is optionally substituted with one or more groups independently selected from the group consisting of halo, cyano, oxo, NR^{r}R^{s}, and aryl, wherein each aryl is optionally substituted with one or more groups independently selected from the group consisting of halo, hydroxy, (C₁-C₆)alkyl, cyano, (C₁-C₆)alkoxy, (C₁-C₆)alkoxycarbonyl, carboxy, and (C₁-C₆)alkanoyloxy, and wherein one or more sp3 carbon atoms of each (C₁-C₆)alkyl, (C₂-C₆)alkenyl, and (C₂-C₆)alkynyl is optionally replaced with O, S, S(O), S(O)₂, or NH; or R¹ is —C(═O)R^{y};
R² is —C(═O)OR^{f} or —C(═O)NR^{g}R^{h};
R³ is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R⁴ is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R⁵ is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R⁶ is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R⁷ is H or (C₁-C₆)alkyl that is optionally substituted with (C₁-C₆)alkoxy or (aryl)(C₁-C₆)alkoxy;
R^{a} is H or (C₁-C₆)alkyl that is substituted with —C(═O)OR^{k} or —C(═O)NR^{m}Rⁿ;
R^{b} is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy
R^{c} is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy
R^{d} is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy;
R^{e} is H, halo, hydroxy, (C₁-C₆)alkyl, cyano, (C₁-C₆)alkoxy, (C₃-C₆)cycloalkyl, (C₃-C₆)cycloalkyl(C₁-C₆)alkyl, (C₁-C₆)alkoxycarbonyl, carboxy, or (C₁-C₆)alkanoyloxy
R^{f} is selected from the group consisting of H, (C₁-C₆)alkyl, (C₃-C₆)cycloalkyl, (C₃-C₆)cycloalkyl(C₁-C₆)alkyl, and aryl(C₁-C₆);
each R^{g} and R^{h} is independently selected from the group consisting of H, (C₁-C₆)alkyl, (C₃-C₆)cycloalkyl, (C₃-C₆)cycloalkyl(C₁-C₆)alkyl, and aryl(C₁-C₆); or R^{g} and R^{h} taken together with the nitrogen to which they are attached form an aziridino, azetidino, morpholino, piperazino, pyrrolidino or piperidino, which aziridino, azetidino, morpholino, piperazino, pyrrolidino or piperidino is optionally substituted with one or more groups independently selected from the group consisting (C₁-C₆)alkyl;
R^{k} is selected from the group consisting of H, (C₁-C₆)alkyl, (C₃-C₆)cycloalkyl, (C₃-C₆)cycloalkyl(C₁-C₆)alkyl, and aryl(C₁-C₆);
each R^{m} and Rⁿ is independently selected from the group consisting of H, (C₁-C₆)alkyl,
(C₃-C₆)cycloalkyl, (C₃-C₆)cycloalkyl(C₁-C₆)alkyl, and aryl(C₁-C₆); or R^{m} and Rⁿ taken together with the nitrogen to which they are attached form an aziridino, azetidino, morpholino, piperazino, pyrrolidino or piperidino, which aziridino, azetidino, morpholino, piperazino, pyrrolidino or piperidino is optionally substituted with one or more groups independently selected from the group consisting (C₁-C₆)alkyl;
R^{p} is a detectable group; and
each R^{r} and R^{s} is independently selected from the group consisting of H, (C₁-C₆)alkyl, (C₃-C₆)cycloalkyl, (C₃-C₆)cycloalkyl(C₁-C₆)alkyl, and aryl(C₁-C₆); or R^{r} and R^{s} taken together with the nitrogen to which they are attached form an aziridino, azetidino, morpholino, piperazino, pyrrolidino or piperidino, which aziridino, azetidino, morpholino, piperazino, pyrrolidino or piperidino is optionally substituted with one or more groups independently selected from the group consisting (C₁-C₆)alkyl.

3. The compound or salt of claim 1, which is a compound of formula (Ib): 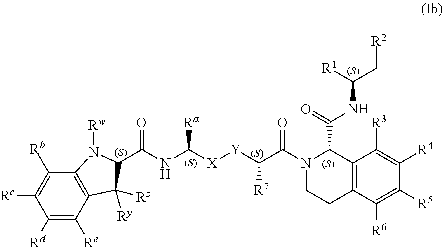
or a salt thereof.

4. The compound or salt of claim 1, which is a compound of formula (Ic): 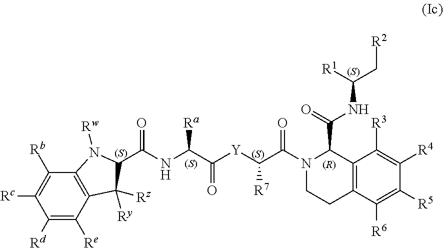
or a salt thereof.

5. The compound or salt of claim 1, wherein R¹ is selected from the group consisting of —CHO, cyano, —C(═O)CH₂O-aryl, —C(═O)CH₂S—CH₂-aryl, —C(═O)CH₂O—C(═O)-aryl, —B(OH)₂, 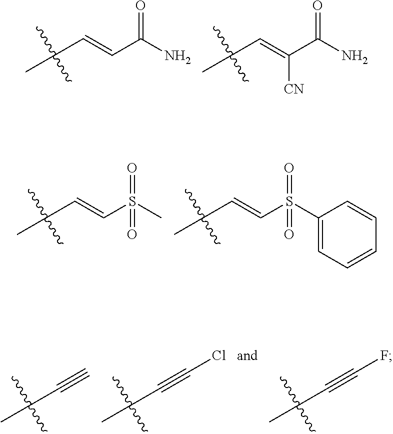
wherein any aryl is optionally substituted with 1, 2, 3, or 4 halo.

6. The compound or salt of claim 1, wherein R² is —C(═O)OR^{f}; wherein R^{f} is H, methyl, ethyl, or isopropyl.

7. (canceled)

8. The compound of claim 1, wherein R³ is H, methyl, ethyl, propyl, isopropyl, F, Cl, cyano, or hydroxy, R⁴ is H, methyl, ethyl, propyl, isopropyl, F, Cl, cyano, or hydroxy, R⁵ is H, methyl, ethyl, propyl, isopropyl, F, Cl, cyano, or hydroxy, and R⁶ is H, methyl, ethyl, propyl, isopropyl, F, Cl, cyano, or hydroxy.

9-18. (canceled)

19. The compound or salt of claim 1, wherein R^{a} is propyl that is substituted at the 3-position with —C(═O)OR^{k}; wherein R^{k} is H, methyl, ethyl, or isopropyl.

20. (canceled)

21. The compound or salt of claim 1, wherein R^{b} is H, F, Cl, cyano, or hydroxy, R^{c} is H, F, Cl, cyano, or hydroxy, R^{d} is H, F, Cl, cyano, or hydroxy, and R^{e} is H, F, Cl, cyano, or hydroxy.

22-24. (canceled)

25. The compound of claim 1, wherein R¹ is —C(═O)R^{p} and R^{p} is a detectable group selected from the group consisting of:

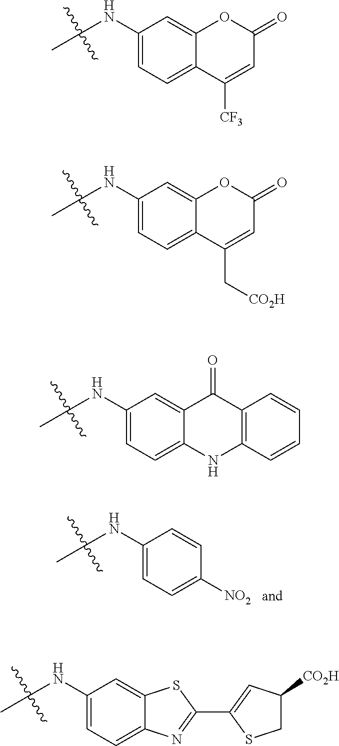 [SMILES: *Nc1ccc2c(C(F)(F)F)cc(=O)oc2c1 ; *Nc1ccc2c(CC(=O)O)cc(=O)oc2c1 ; *Nc1ccc2[nH]c3ccccc3c(=O)c2c1 ; *Nc1ccc([N+](=O)[O-])cc1 ; "and" ; *Nc1ccc2nc(C3=C[C@@H](C(=O)O)CS3)sc2c1]

26. The compound or salt of claim 1, which is: 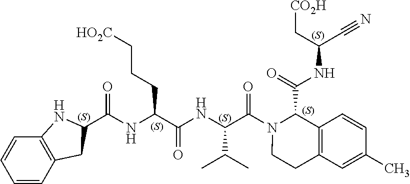
or a salt thereof.

27. The compound of claim 1 that is selected from the group consisting of: 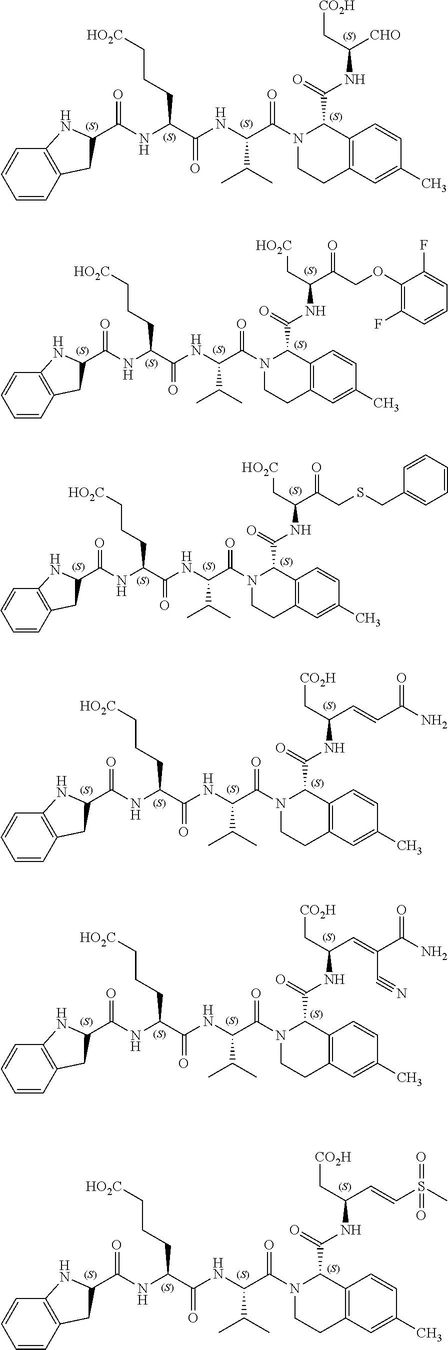 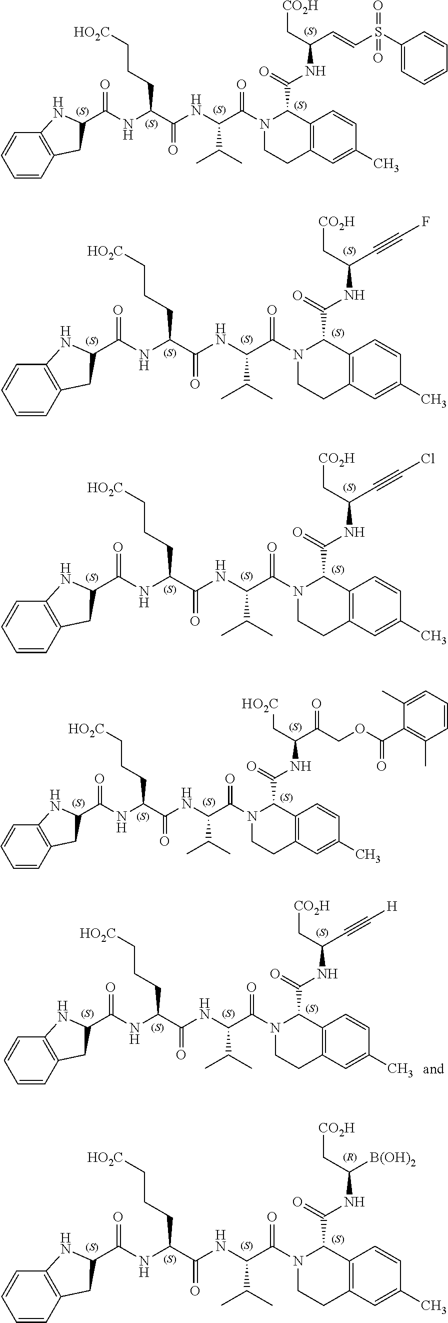
or a salt thereof.

28. The compound or salt of claim 1 that is a compound of formula (Id): 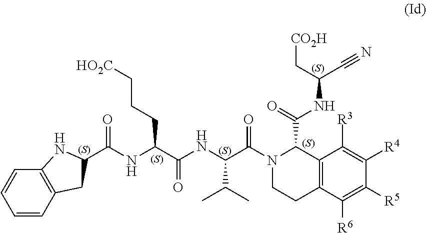
or a salt thereof, wherein:
R³ is H, methyl, ethyl, propyl, isopropyl, F, Cl, CN, or hydroxy;
R⁴ is H, methyl, ethyl, propyl, isopropyl, F, Cl, CN, or hydroxy;
R⁵ is H, methyl, ethyl, propyl, isopropyl, F, Cl, CN, or hydroxy; and
R⁶ is H, methyl, ethyl, propyl, isopropyl, F, Cl, CN, or hydroxy.

29. The compound of claim 1 selected from the group consisting of: 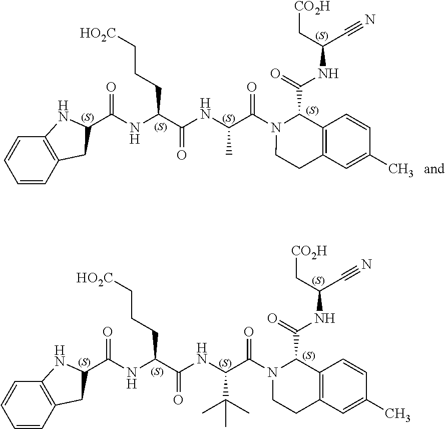
or a salt thereof.

30. The compound of claim 1 selected from the group consisting of: 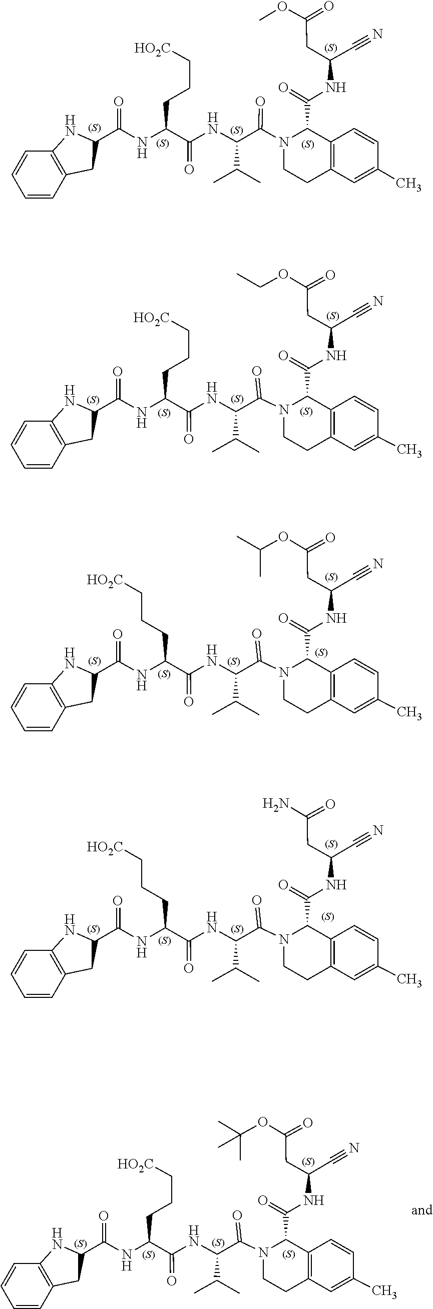 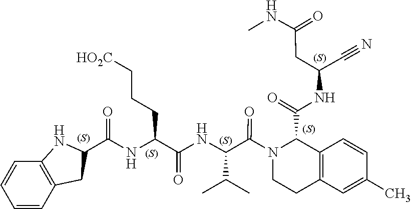
or a salt thereof.

31. The compound of claim 1 selected from the group consisting of: 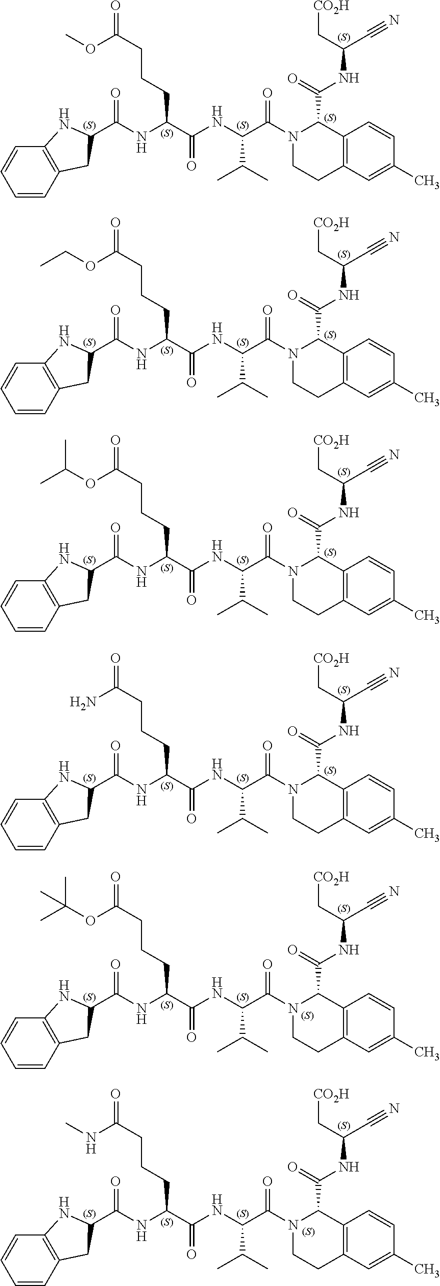 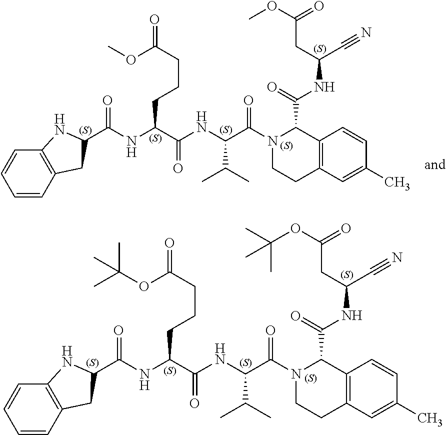
or a salt thereof.

32. (canceled)

33. The compound of claim 1 that is selected from the group consisting of: 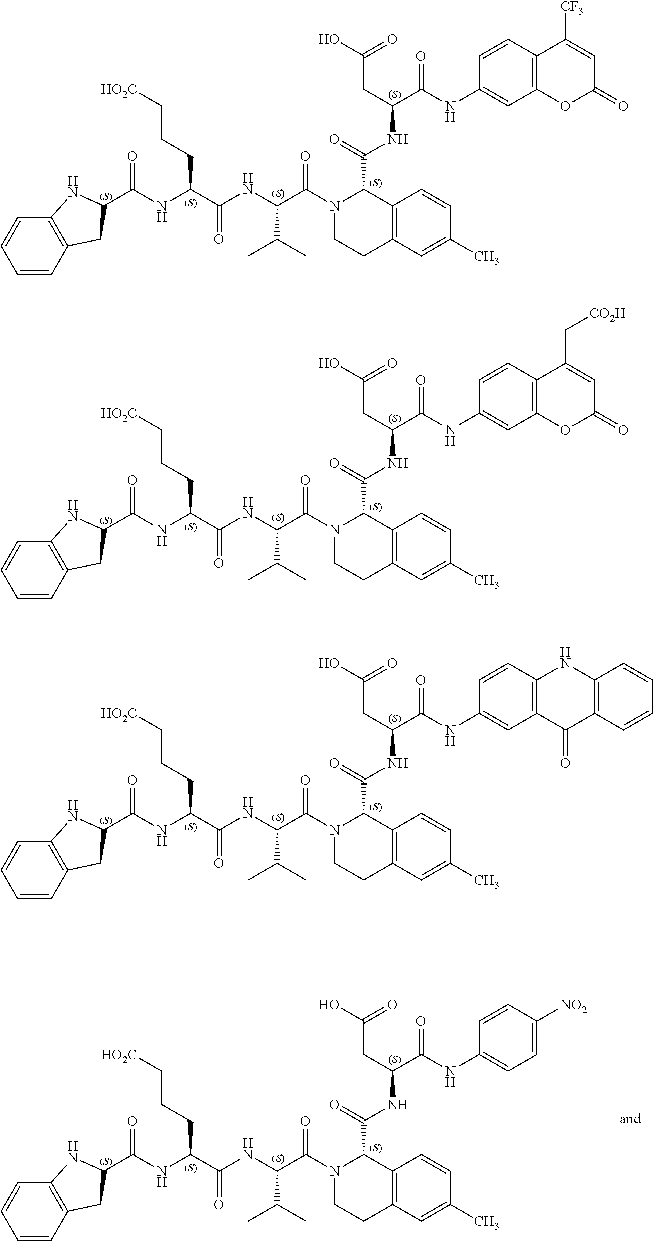 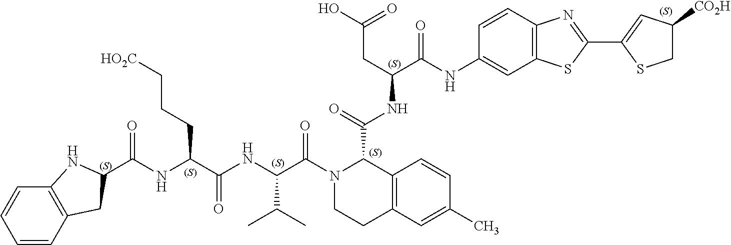
or a salt thereof.

34. The compound of claim 1 that is selected from the group consisting of: 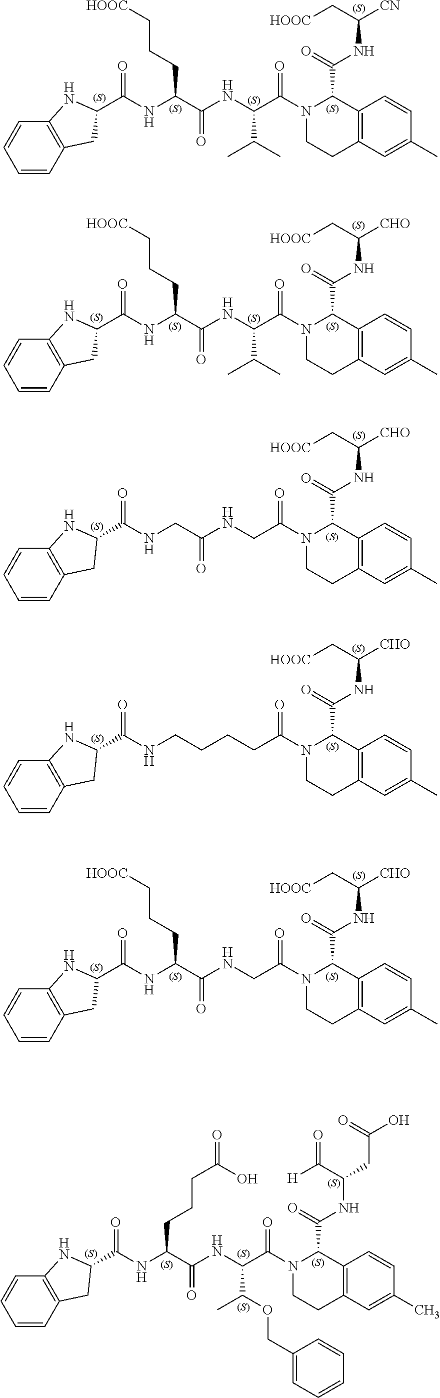 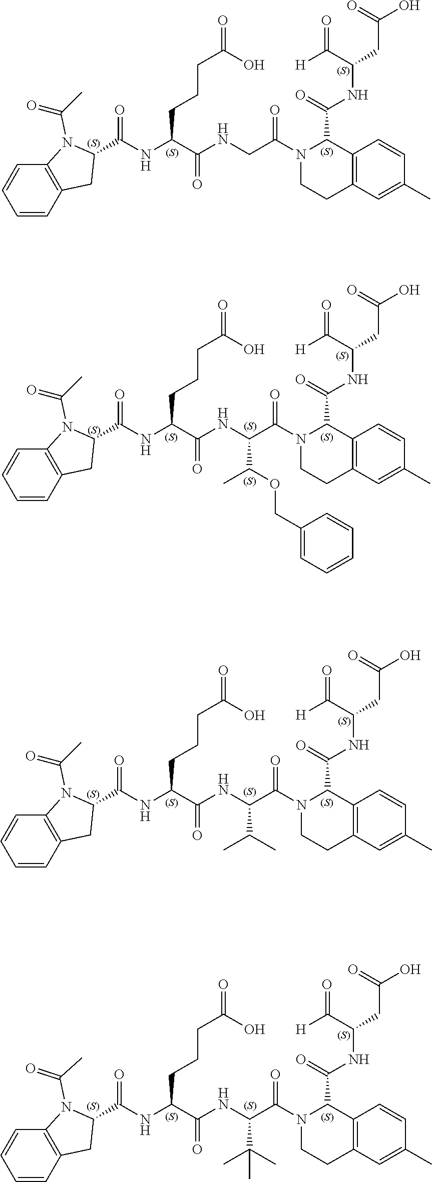 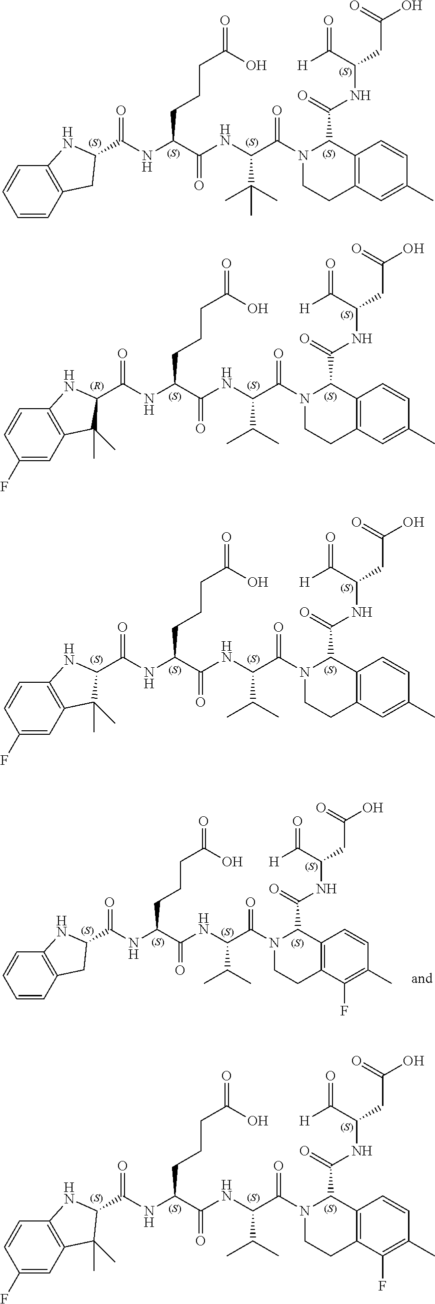
or a salt thereof.

35-45. (canceled)

46. A pharmaceutical composition comprising a compound as described in claim 1 or a pharmaceutically acceptable salt thereof, and a pharmaceutically acceptable excipient.

47. A method for treating a neurodegenerative disease, a liver disease, or cognitive dysfunction in an animal comprising administering a compound of Formula (I) as described in claim 1 or a pharmaceutically acceptable salt thereof to the animal.

48-50. (canceled)
